SEQUENCE LISTING (120 sequences)

SEQ ID NO: 1 - DNA, 1050 bp, Homo sapiens
  source 1..1050: mol_type = genomic DNA
tgcagttcctagccaggctcaatggtcctttctttctctgctcccagcaacgaaaacacatcgaaaagaagaccccacaaaccccaaccccgaaaatgctccccaggtagagaccctcccgcagcagaggctcggctcaccctgccgctggcccaggccactgcgagcagcgccgcgagcgcacagagcacgatccccagcagcagcagcagcagcgggtactgctggctcaggctgtagtaggtctcgtagaagaggtcttcgctggggggcggccgggggctgaagaggcgggccatgatctccccagccccgagccccggggctggctagggccgggcgccgggttacctccttcggcccggcgggccccacctgagcttttctcacccgctcaaagccgctaccaccccgcgcccccaacctcgtggcaatcccgtctcctttttcaggccctccctgcggcctcccagcccgctcccagctggcgatgaggggatccctcagtcctttcctcctcctccctcaaacccggattgtagaggtgccctagaaaagcctcatcgccaggagggcaggatttggggttggtgcgagggagccccgggttcccctgatccccgaactcactgcccgcggcgctggcgcagcggagtgggctaggtccggggagggagccggggacctgatggcggagtcacgctcgccgctgcgctctggctcagagtcccgggcgacaggcgcttccctcccggcgccgcggccccctcccacttccccgacgggacagcccggccccttcctgtgccaaagccggactcccctggtctcctcccgcacttggttttgtctctcctaacaccatcgcctccccgcttcagccccttcagggaagggcaatgtggaccctgggagagatgcgaaggggtgggctggggtggacccaatgggatgtgtccaaatggaggtgaacgtttggtagtggggagaccgagggcggggggctgaagcctggcgagaatgtagggaagaatgtgggtcctggtgggg

SEQ ID NO: 2 - DNA, 799 bp, Homo sapiens
  source 1..799: mol_type = genomic DNA
gttggaggggagttactaatgttcccagacttaaatccagctggaacaccacctaaaatatgcagtaacataagaccatcaaaagcaatgtcccaggacttacaatgtttgctaagacgcaagagggtgtgacacagacgctaagcgccactggcgaggagatgaaggggtcgtcttcatcttcgccggatgatttccgcccacatagagggcgccagtgacgcccacacacgtgctggtgtcccgggaagagttcctggcaaagagctcaggaacgttggatcttaatcaaggctttctccgtcggggtggatgggttggactttaggctccagcaagccccgccccactcggcgggtcggtgccgccgggtcccaggtgcccgctacttcccagaacctccgcctcccgctccgggccctcgaaccagcgcggacaccacaatggaccgggcgtccgagctgctcttctacgtgaacggccgcaaggtgagcgcccgcgggcttcctctgcccccagacctgcggccagggccggggcagagaggagcccctgccgttcgtcccatcctttcgtgcccgccgtttaaggcactcaggcacggactggctttctcccgtaacagcggctttgccttcgcattcccacccctgcctccggggcagctaaaggctgcgttttcagggactgtcctggatctttgggcccccaccttccttatgttcccctttcctctccttccaaggctcacaagctgatttccaaccacacgccctccctaaacagtaagataaaaagt

SEQ ID NO: 3 - DNA, 600 bp, Homo sapiens
  source 1..600: mol_type = genomic DNA
ccgcgctccgcagggtctgcaagggaattactgggggtctcagaactcctccttccagcccgcggcctgagggtcccgcccccgttccccgcaccctcctggcctgaccccggcccggcctggcccgcctggcgccccacttccccaccctgtaagtagcccgaggtcccggctggggtcttgggacacccctccaggctgcagcctccaccgtccctgacgtgcactcactcaggccggacgccagcgcctgttcgttggcccacatggcccactcgatctgccccatggcgacacgaacccggctgggacactgctaggcgcgcactgccgcccctgcgctcccggccgaaccccgccccgcccaggccccgcccccagccccccaggtgaccgcggctgggcggagacccgcatctgtagggtgcagggctgtcccggagccttctgcccccgccctctctagccacgccgaggcataggaggggccctgcccggcagccccagcccaggcggggagttcctgcgcttccccagggcttcccaaacttcagctctcaaacactgacaagtgcccctgcccaatgctccggacccaac

SEQ ID NO: 4 - DNA, 2340 bp, Homo sapiens
  source 1..2340: mol_type = genomic DNA
cgctctgtcgccctggctggagtgcagtggcgcaatctcagctcactgcaacctccgcctcctgggttcacgccgttctcctgcctcagcctccctagtagctgggactacaggcgcccgccaccacgcctggctaattttttgtatttttagtagagacagggtttcaccatgttaggatggtctcgatctcctgacctcgtgatccgcccgcctcggcctcccaaagtgctgggattacaggcgtgagccaccgcgcctggccagaagctggcttcttgcagggacagcagggccgcaaggctctgtgactctaggccccatcacacccggccctgtcccacttcctctttcggccagaaccccagaactccagtcattcataggccgggtcctcctcgggggaaccaggcctgccccctaggaatcagggtatccacgcctcctcggcccgctctcggagcctgggcctcaacacctgcagattggctcccgggccaggacctgtcgcttgaacatcagtggccgccgccgtccaatcacagacgtacaggggcgggctggcgggcctgttgggccagcaggaggacggcgccgggccgaggccgctggacccgccgttggaaccacagctgggtgaagtcagtgctgctaggtctttggagccagaccgaagggagggtcgcgcgcccctgggtgggtagcaccaggcggcggccgtcggccgggcctcccgaagcctcctccagccgctaataccgcccgcgcctccctcctagtcctagacctggcattgcctgtccctcttcttcgtcttccagaccctcctgacccgccatattaggtcccggccccgccctccgtctcattgcccattgcccaccccaccccaccgccccctccccggcctaccgtcagctcattggcccaggctcccgccttcttcgcccaatgttaggcctcacctcctctaggatccaccctctccccgcccacccgcataagccaggcccaccattccaactctttctcctttttggcttgtaggccgcacctactctgcatatgccccgcccctcactccaggtgtcgcccacttaactgaagatccaggatcccctccttaaggtttgtctgggctcggaattgccttttttttttttcttttttgagacggagtttccctgttgtgacccaggctggagtacagtggcgcgatctcagctcactgcaacctccacgtcccgggttcaagcgattctcctgcctcagcctcccaagtagctgggactacaggcgcccgccaccacgcccagctaatttttgtatttttagtagagacggggtttcaccacgttgcccaggctggtctcgagctcttgatctcaggtgatccgcccgcctcggcctaccaaagtgctgggattacaggcgtgagccaccgcacccggcccggaattgccgcctttagaccctcacccacccgctgctgagtcttcctgggccccgccccggccctaagccccgcacaggacctgctaggccccgcccatttatttcccccctttccaacctcttcctccccagcctccgcaccctacccttgtttcctgtccctgtcgcgcccaggtgtttacctggcactcaggtgagtggtgcgctctggctgttttctgtcggagccgcccgcctcttccttcagcgcgtcccacaaatcccgacggcacggaggggccccaggccaagggcgatgggcccctgagccctgacaccgcttcgccgctgctgcaggtgcccctggccggcagcgccgccgtggtcccggagcgcggcgacatgccggagctggtggtgaccgcgctgctggcgccgtcgcgcctgtcgctgaagctgctgcgcgccttcatgtggagcctggtgttctcggtggcgctggtggccgcggcggtctacggctgcatagcgctcacgcacgtgctgtgccggccccggcgcggctgctgcgggcgccgtcggagcgcgtcccccgcctgcctgagcgacccctcgctgggtgagcacggtttcctgaacctcaaggtgatcgcacgggggcgctatcggggctgcggggtcgcgtggggtcgagacggttcaagcctcgggagagagggttccccaggcttcaggctgtgccctccccctagagctcgggcctgcgtctgcactatgtctcggctggacgaggtaacggacccctcatgctgtttctgcacggcttccctgagaactggtacgtctggggccaggggcctgaggcccacgggactcgggggcaggtggagcttgggctcccaagag

SEQ ID NO: 5 - DNA, 1250 bp, Homo sapiens
  source 1..1250: mol_type = genomic DNA
agcaagggacaagaggagcagaggacaggtgatggaaatcctgcagctttaggctccattctgccatctacatcccagcgcagggtgaagcctgagagcccaaatggccaactccacagggctgaacgcctcagaagtcgcaggctcgttggggttgatcctggcagctgtcgtggaggtgggggcactgctgggcaacggcgcgctgctggtcgtggtgctgcgcacgccgggactgcgcgacgcgctctacctggcgcacctgtgcgtcgtggacctgctggcggccgcctccatcatgccgctgggcctgctggccgcaccgccgcccgggctgggccgcgtgcgcctgggccccgcgccatgccgcgccgctcgcttcctctccgccgctctgctgccggcctgcacgctcggggtggccgcacttggcctggcacgctaccgcctcatcgtgcacccgctgcggccaggctcgcggccgccgcctgtgctcgtgctcaccgccgtgtgggccgcggcgggactgctgggcgcgctctccctgctcggcacgccgcccgcaccgccccctgctcctgctcgctgctcggtcctggctgggggcctcgggcccttccggccgctctgggccctgctggccttcgcgctgcccgccctcctgctgctcggcgcctacggcggcatcttcgtggtggcgcgtcgcgctgccctgaggcccccacggccggcgcgcgggtcccgactccactcggactctctggatagccgcctttccatcttgccgccgctccggcctcgcctgcccgggggcaaggcggccctggccccagcgctggccgtgggccaatttgcagcctgctggctgccttatggctgcgcgtgcctggcgcccgcagcgcgggccgcggaagccgaagcggctgtcacctgggtcgcctactcggccttcgcggctcaccccttcctgtacgggctgctgcagcgccccgtgcgcttggcactgggccgcctctctcgccgtgcactgcctggacctgtgcgggcctgcactccgcaagcctggcacccgcgggcactcttgcaatgcctccagagacccccagagggccctgccgtaggcccttctgaggctccagaacagacccccgagttggcaggagggcggagccccgcataccaggggccacctgagagttctctctcctgagcaggagaaaggagggtggtttccgtgggggctcatccaa

SEQ ID NO: 6 - DNA, 550 bp, Homo sapiens
  source 1..550: mol_type = genomic DNA
ttcagaggctggactagagaaggcgcgttcagccgacggtgcgggatacactgccgaggaagtccgcggcgaggggcgtggttggggggtgtgtattacacggtatcccccccttgcctttccaagtctttccctctggttccgcctccttttcaggagtgacataccgttgcccaatcagagcctcaggcctggcgtggcacagccaaccgcgcggcgtggggaggagctggggagggggctcgcaaggccggggagggggctaggggcctccgagggctccggagcggcgactggcgagccatggcgctggggctgcagcgcgcaaggtactgaagttcgggggcaggagttctgggaattggggggctctcctcctggagacccctgagctggattgttggggggggtggggttcgcgggtgcgagccaagaacgccccggggcgcgcagttggaggcacatccccaccgcactctccacccttagggactgcaggggtgggggattccctacgtctctggctgccccgcccctctggccaagagcg

SEQ ID NO: 7 - DNA, 800 bp, Homo sapiens
  source 1..800: mol_type = genomic DNA
ttgcattgtccatctgtcactgagtgacctggtcctgcgaagcccggcgtgactgtgccaactttctcacttcctccatggggccggagaagaaaaatgatatgaatgtacagtgcgcaagagggggggcgggagggcggaggacgctggaggaggggcacgtgacggtgtcagccaatggctgagcctcctgcaaaagtttgccggcttccgcagtgatggatcaccgttttagtggcatttaaatccccggcgctccgccgtctaggtgacgcgcagtcgcccccccaggcagcctaggcggcggcagctgctgcggcgactgcaaaggccgatttggagtgctggagcgaagaagagcaaaagctgcgttctgcgcgcgcccgactccgctgcccgccccgccaggcctccgggaggtgggggctgggaggcgtcccccgctcccgccccctccccaccgttcaatgaaagatgaactggcgagaggtgagaagggaagagggctcccggctctctcggggcgggaatcagtgggccagagctcgccgggtggccgcaagtacgccggcccagcccgcagcgcgcccagccggaaggcggggaatccggctgacaccgcgccccgggttcccaggccacctcctctgttctgaggctgggctgggagaccgtggggctgtgaggagcgcatagaaccgtggtggagggcgaggctgggccaccggctcttcaagctcggaatggagggggaagagcgcagagggctggctgggaggaactcgggtgggcgtgaagga

SEQ ID NO: 8 - DNA, 800 bp, Homo sapiens
  source 1..800: mol_type = genomic DNA
gcaggggagggagaacggcaaggagagctccgcagggctgggagaaatgagaccaagagagactgggagagggcggcagagaagagaggggggaccgagagccgcgtccccgcggtcgcgtggatttagaaaaaggctggctttaccatgacttatgtgcagcttgcgcatccaggggtagatctggggttgggcgggcggcgccgggctcggctcgctctgcgcactcgcctgctcgctgctggcaggggcgtcctcctcggctccggacgccgtgccaaccccctctctgctgctgatgtgggtgctgccggcgtcggccgaggcgccgctggagttgcttagggagtttttcccgccgtggtggctgtcgctgccgggcgagggggccacggcggagcagggcagcggatcgggctgaggagagtgcgtggacgtggccggctggctgtacctgggctcggcgggcgccgcgctggcgctggcagcgtagctgcgggcgcgctctccggagccaaagtggccggagcccgagcggccgacgctgagatccatgccattgtagccgtagccgtacctgccggagtgcatgctcgccgagtccctgaattgctcgctcacggaactatgatctccataattatgcaactggtagtccgggccatttggatagcgaccgcaaaatgagtttacaaaataagagctcatttgttttttgatatgtgtgcttgatttgtggctcgcggtcgtttgtgcgtctatagcacccttgcacaatttatgatgaattatggaaatga

SEQ ID NO: 9 - DNA, 550 bp, Homo sapiens
  source 1..550: mol_type = genomic DNA
cagcaagacgcaaagagaaaagaccaaggcccccgccgccgccgtctgtctagactcaagcgactgaaggggccaacagagctggtgtttaaagtagaacctgcccagtccaacagcccgagcagggagcgatttcggggatcgcgggaaggaacgcacttcgccaagggagggccgggtgccctcgccaccggctcattcctgctccggttttgcccgatgcgcgtccaggaggttctggcaggacgcactgcccctctgccccggccaaggaggatgcggatactgcccgcaaggcttcggcctttatggacccaagtcagccaactgggccgagtcctgcggacaccgaaacctccctttcgtttccaggcttccttctcccctcttgccctctgtggtctgatttaaaacgaaaaggtcggataaaatcaggctttcaataaggcttctttaactgtgtgttctctattcattggttctctacttatttgactgaaaagacacaaatgcactaggttatgtgagataattttcacagaaatactca

SEQ ID NO: 10 - DNA, 550 bp, Homo sapiens
  source 1..550: mol_type = genomic DNA
gacggggttgtggtggcccgccagcgggggatgccaggccacctcccccagcggcacgcagcccctctcttaattagatcggttttcccctggtgtccgggagagcggtcccggcagaaaggtcggtatgggggtgtgcgctgttccgcataaccactgcctcccatgtcctcctcgagggccgaaccgagagggtgctggcagggctggatcccacgggtgtccgcaggagacaaaggcgaattccggagaaaaggctggggctgagaaaggcgctccgggagcggctggcagggcaattcggcaggctgcaccgaagccgagtgcccggagggacttgccgcccggaagggggtgtgtgggggcgctgccgtgaagatggatgagggaaaaggtttttgatatcagcagaagggaaaacgcctggagtggccgaacacttttagttgcccagcaggaataggagacgggtactcagctccccaaggctgcgcaatatcccagctttgcccgctcctgccctcgtgttcggaatatgctggcggtgtga

SEQ ID NO: 11 - DNA, 1050 bp, Homo sapiens
  source 1..1050: mol_type = genomic DNA
ccggtgccttgggtccagagttccggacccccaggaaatcgcaggtcgcggggagcagcagagagaagggggtgggagagggggaaaagaaggcaaggaaaaaaaagcccctgcgcattgatccgcgccgtatttttgggtaaatacgatcacgtgggggccggggagccaatgagctgtggggaaaaggctggaaaaataattacctgacttgattgttctgtgagcagataaaaagtacatatacagttcatacaataatcttatgtatgtaaaaccccgttacgatgtcggcgacggggcccatcagtaactattacgtggactcgctcatctctcacgacaatgaagacctcctagcgtccaggtttccggccaccggggctcatcccgccgccgccagacccagcggtttggtgccggactgtagcgattttccgtcctgtagcttcgcgcccaagccggcagtgttcagcacgtcgtgggcgcccgtgccctctcagtcgtccgtggtatatcacccgtacggcccccagccccacctcggcgccgacacgcgctacatgcggacttggctcgagccgctgtccggcgccgtctccttccccagcttcccggccgggggccgtcactacgccctcaagccggacgcctaccccgggcgccgcgcggactgcggcccaggggagggccgcagctacccggactacatgtacggctcgcccggggagctgcgcgaccgcgccccgcagacactgccctcgcccgaggcggacgcgctcgccggcagcaagcacaaagaggagaaggccgacctggaccccagtaagttgggagcaattttcctttacaaccggcgcgggaggggaggggaggacgggcgggggcagccgaattacagccctcccgaaccgtgcagggagcgcgggagaggggcgagggggcgaggactcaataaaagcgaattaccttggggagctttcaggggcaggaggtctgagaatggggcccagggagacaggttgggagagggccgaggggagctcctct

SEQ ID NO: 12 - DNA, 800 bp, Homo sapiens
  source 1..800: mol_type = genomic DNA
tctctttcaacttggatgggctgactctacccgtcggtgatttacgacgattgcagcgctagtcacagcctggcgcctggtgtcccctcccttcccaagccccctcagcttttccactgccaccggcgtacaagcaagtgccgagccggcctccgcaagtcggactagcctcccggcgtccgaggccaccacgggcagcagatttttggtccccagcgaggctgcgcgcgttcgtcccgcctccgaccgccgagcagagctgctagcagaagcaggcgccggtcactttatataatcctgctgctcgcagggtgcaagagcgggaaaagtgcggagtagggaattcttttgctgcgctgcctcctacgcggagcctgctttccactgctgaaaagtgccgggccttgggaagtgtttttcttttcattccttaccgaagcgtttactgccgccgtggtcgcagtcataaattttgctacaaaccacaatgacaggtgcattgatatgcaccgtgagagctccagctgcttaataaccccgtcccctggtcgctgtgagcgccttttatttatttggtatcatattaggtattgatctctagtagcattaagtgcggtgagcaagtatcagggttcggctgctttggaggcgcagcggttgcggcgggccggcgggcccggggaagcgggcggtggccgctcagagaataccttccttccggcaggagaccgtttggccctgtattccgggcctgcggttgggcctccaagctgagttgggcaacttcccagcaccgcaag

SEQ ID NO: 13 - DNA, 1100 bp, Homo sapiens
  source 1..1100: mol_type = genomic DNA
cgggtcctgcccagacctggagtcgcacagatcacggcgggcagtggctcagcgcctaggcggctccaggcctcgaaggaccaggttggggtgctcagggatcagagaggggaggtcgctctgggtccgggtcgcctgctacgcgccttttctgtctcagaagtggcggtgactcggctgctgagtccgcggaacgagccacggaatggtggtggtggcggggttttctgaggtgactggccagagctgagagtcgcggcttccacctttgggccggagcgggtcctcgccctgggaggagctgggcgtcggcctccgcggcggggaggccgccttgccggggtgcatgaggctgcggagtactccgcgggcccgggaagctaggggtaccctcagcctctgctgctccacggcagtctccagagacgcttctaagagaggcagtttctaaaatttccagctcccggaccagtctggcggaaggcccagccagggtcagaggtcgttgtggggagagactctcaacgcccccaacccactgagggcggccaggccaagactgagtcggcccggagctgcgaaaatgtgttctttccttccgccccacacccatccgcgtctgccccaggaatggggcccaggtcccaagcctcctgcgcccttccttccagcccccaggcttggctgcgctccgggactgggtggcgtgaaagtttcagcctcaatcagtacaagcttccctcggggtcacgtgaacaaatatgcttgcatttgaaggcagcgtctgtatttcccgactatgagggggtttccggggctctctccaaatccagaaacgaccacgttccgcaagcaaaacaaatcccaagctctggggggcctgggagggctgggcagaaacccaggagtgggtgggggcgcgggtggctgccgctctgggcccgagagcggacgggcgggcgggtggaccgatgggcgcgcagcgcaggcgaagccagctcggggactacgaactcgttcctcctgcgtttattggtagttgaacctcagcctggttccgttctaccgggaattccgtgtgctcgagtatatggccgtgt

SEQ ID NO: 14 - DNA, 800 bp, Homo sapiens
  source 1..800: mol_type = genomic DNA
gccatagcaagctacttgattacacgtatgttatttagttaaatttgtgaaaattatgagatgctcaccaacccggtgataaacttgctccctcgccattggctggcctggtcacatggctgcccaactttattcagttgacagcaagtaggagggccctatggaaggagaaaaaaagacaacacgagaaaaattagtattttctaccttctgaaattaatggtcatgagttcgtatatggtgaactccaagtatgtggaccccaagttccctccgtgcgaggagtatttgcagggcggctacctaggcgagcagggcgccgactactacggcggcggcgcgcagggcgcagacttccagcccccggggctctacccacggcccgacttcggtgagcagcctttcggaggcagcggccccgggcctggctcggcgctgcctgcgcggggtcacggacaagagccaggcggccccggcggtcactacgccgctccaggagagccttgcccagctcccccggcgcctccgccggcgcccctgcctggcgcccgggcctacagtcagtccgaccccaagcagccgccctccgggacggcactcaagcagccggccgtggtctacccctggatgaagaaggtgcacgtgaattcgggtaaggctagggtccagtaacctttctgtccacatcccagcccgttagcctgggtcctctggaagggggtgcgagtaggtgggggcgtgtggagcttccatgggcgccgcaattactctccccataaatttttatagctgagggagca

SEQ ID NO: 15 - DNA, 2050 bp, Homo sapiens
  source 1..2050: mol_type = genomic DNA
cgcgaagcggccctgcccggggagactcgctgaggcagggctgaggcggcgggcgggagcaagctgctctagcatttgggttctgccctgtggcgtgttctcttccagggcctttccagcatcatcggagaagacgaagcaccctggccgccactgtccgtgctgcgccaactcgcccggccgcccgcccttccgagggcaggcagaagcccctctgtgtcctccaccgccgcgccccggctcgcccctcgggccgcggcgtgtgcccagcctcacgtcggggtgtgtgtggccgcgcgggcgtgtgtgagtgtggcagggggagggggccctccgatctgctccatccgtccgttttattagggacacattaatctataatcaaatacacctcataaaatttttattgaaaggcataatatcattacagaggtcttccacctgttttaaacaacacgacaagctgtgagcaagcgtgtgtgtggggatgtgtggggaggggtgggtgtgagtagggagagaggcgaggggagaacagctcccctcgggcgctaggggccgccccgagggcccgcctgcctcgggcgacaccggcctggcgcccccgcggccgctccgtgtgccctggactcgccgcccgcggctcggaagctggagagtcagcgacggggcccgactgcgggaccgagggctgcaagaagaagcgaacaaatagtccccagcgcctcctctggatgcggtcgcgtctgtggtcctggcagccgctgggcgggccaggccaggtcgggccgggccgagccgggcacatggacctgggcctgcgggctctaattgcggcgcttatgttgatgattttttttttaatcacagcagcccccagtttagcggactgatttactcccggtattggtaaatatgatcacgtgggccgcgcgaccaatggtggaggctgcagcctgcgaactagtcggtggctcgggcgccggcggggagctgctcggcggcggacagtgtaatgttgggtgggagtgcgggacgcctcaaaatgtcttccagtggcaccctcagcaactactacgtggactcgcttataggccatgagggcgacgaggtgttcgcggcgcgcttcgggccgccggggccaggcgcgcagggccggcctgcaggtgtggctgatggcccggccgccaccgccgccgagttcgcctcgtgtagttttgcccccagatcggccgtgttctctgcctcgtggtccgcggtgccctcccagcccccggcagcggcggcgatgagcggcctctaccacccgtacgttcccccgccgcccctggccgcctctgcctccgagcccggccgctacgtgcgctcctggatggagccgctgcccggcttcccgggcggtgcgggcggtggcggtggtggtggaggcggcggtccgggccgcggtcccagccctggccccagcggcccagccaacgggcgccactacgggattaagcctgaaacccgagcggccccggcccccgccacggccgcctccaccacctcctcctcctccacttccttatcctcctcctccaaacggactgagtgctccgtggcccgggagtcccaggggagcagcggccccgagttctcgtgcaactcgttcctgcaggagaaggcggcagcggcgacggggggaaccgggcctggggcagggatcggggccgcgactgggacgggcggctcgtcggagccctcagcttgcagcgaccacccgatcccaggctgttcgctgaaggaggaggagaagcagcattcgcagccgcagcagcagcaacttgacccaagtaagtgcaaaagaaattgccccctgatttattgctgaaacctgtaaggctcgaatgtgcaaaactgatagttttactaacctataaaaacgtctagacgcctacccaagcctaggcgaacaacatgcatccataaaaagagcttcccataaccacctaccctgggcgctcagttagtacggtaaacagagcgcgagcattaaggctttttatgataa

SEQ ID NO: 16 - DNA, 1100 bp, Homo sapiens
  source 1..1100: mol_type = genomic DNA
aatcccatctctactaaaaatacaaaaaactagccgggcttggtggcgggcgcctgtagtcccagctactcgggaggctgaggcaggagaatggtgtgaacccgggaggcggagcttgcagtgagccgagatcgcgtcactgcactccagcctgggcgacagagcgagactccgtctcaaacaaacaaacaaacaaaacaaaacacggtagtgtaaaatggagcggggccgggatggggcggcgtaagaggaggagaaagaacgcggcgaagagtccacggaagagcgaggatccgggtggcagaaatcggacagggcctgagtggggcgggtcacccaggggccgagccagaagaagggcccagctgacttgggggcgggccgtgctccggagcgctgggtgggtgctggggtctcgggggaggagaggtgcgcggggcttggtgacgggaacacattctcctcccgcccgtgcctcccaccggcgcctctccccgccccctgcccccgaccttaccccagagttgcgccatcagcagcggcagcagcttcgccagagcccgggcgccagaggcggcggagaggtggaggtgcggagctctcatggccaggatctgctggggtgtgtgcaggggcgggttaaaacagatgctccgttagagacccccacctcgccgcgctcatccgcccagcctgggccaccccagcccgcaagcacccttttgacctgcagccgataaccccaggggctggcagacgggagattcgggctggaacagcggtaatgggcacaattaccctaatgacgcccctcgcggcatcttcccgtcctccctgtgcccgagtggagcgctctccgcgccccagctaccctggctgcagccacgccgcgcccgaggtttccccctccttcacgcgcggggtggggatccgaggctcggagccagtgggagcctctttctcaacctcacagcggggggacttccgcgtcccgcaggtggagaaaccgaggctctaagccggctcctgcctgtggcccgggggtccccacccatcctctcgcaatccaccccaacccgggtggggtgcaggtagcttca

SEQ ID NO: 17 - DNA, 1300 bp, Homo sapiens
  source 1..1300: mol_type = genomic DNA
gtggcctgaccagagccacacgaggctcttctcactgggcgaggctctttgaggaaccgagagttgctgggacccagcccgccctcgagagagcaaacagagcggcgctcccctcccccgaccccggccctttgtccggaatccagctgtgccctgcgggggaggagcgggctcgcgtggcgcggccccagggccccggcgctgattggccggtggcgcgggcagcagccgggcaggcacgctcctggcccgggcgaagcagataaagcgtgccaaggggcacacgacttgctgctcaggaaatccctgcggtctcaccgccgcgcctcgagagagagcgtgacagaggcctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggtaatatttggaggcctccgagggacgggcaggggaaagagggatcctctgacccagcgggggctgggaggatggctgtttttgttttttcccacctagcctcggaatcgcggactgcgcccagtgacggactcaaacttacccttccctctgaccccgccgtaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctt

SEQ ID NO: 18 - DNA, 1550 bp, Homo sapiens
  source 1..1550: mol_type = genomic DNA
taaagcttccccagagggaggaaaggtgggggcggggcggctgctgaggcccaggatataagggctggaggtgctgctttcaggcctggccagcccaccatgcacgcccactgcctgcccttccttctgcacgcctggtgggccctactccaggcgggtgctgcgacggtggccactgcgctcctgcgtacgcgggggcagccctcgtcgccatcccctctggcgtacatgctgagcctctaccgcgacccgctgccgagggcagacatcatccgcagcctacaggcagaaggtaggcagtgccgcgtgccgcgccctgctgggcacccccggggcgcctccgccgcgtccagccagcggactcgggaagtgctgtgggttgggggctgcggctccgagccgggtttgcagccgcccgggcgtcccgagcccagggcctagctctgcgggtgtctccgcgtcagcaggctcggggtgcagcgttggtggctgggggcgtatccacggccgagtcgggaagggattctagcgttcagggtgtgtcctcgacggggaccattgtctctgggttttggtttgggattgcgcggagcgcagcgcggaagggtgggagcttctaatctccagtcttgtgaagttgcttatcccggagcctgggtctgcgcatctgtaggataggtgtaataaataacacctcgcctatcagactgtggaaagcgcgagatgacaatgcgcgcgaaacgctcagcgcagtacccggcacagccacagtcaacggtcgttggtattactgtaatggtttggtcttggcgatttttttttctttctgcgagtgagggtgaatgggtcccggggtgtgacgtcgggagtatcggcagctgagctggtaacatcggggattcgggctcacggcccggagatcagggatgggctgtcccgaagtcgcgaactgtggcagccttgggtcctccagccgcgccggggaagtgtcaagtgtctcgcttaaccccgggttcggggccatgatttgcaggggagtgggtgtcaaggacggcagggatctgagggtatcgccctcgaggacctggcagcgcgttctgggcacccagcgcggcgagcaggtgggtgctgcggagagggagccccttccgcgcctcaatccacattctgccgcctgggcagccgcggccgcccacgcctccctccgcctgcgggggccagacggccctccctggggccggggcgcaatccacaaacgctaatctgatccgacctgccgcctgcccgccccttgtgacctggtgccgggggcccttcgctcccgcgcctggggtcagacagccggtgaccctctccggaagggtcatctggggaccagccagaccaggggacaccctcgggggcggggcaatgagaaatttgctggagtgctcggcccctcaaccgaaaagcggccggggatgggagggggcaaagaagggagggagcgcttttccagttcactcccttctggaaagttcgagatgtgtgcggtgatggacaggcatctg

SEQ ID NO: 19 - DNA, 850 bp, Homo sapiens
  source 1..850: mol_type = genomic DNA
taaaatcagcgtatttttacatataagcagccacctctgctcatctgtggcccagatacgagtggagtgcgacaagggataaaccattttcgcgcactcttcagcgatggggcgaaagtaacggacctagtcctcgggagctgtccccgccgaccccctctgccgcgacttgacccgcggcgactgcgctgccccttggctgccccttccgctctcgtaggcgcgcggggccactactcacgcgcgcactgcaggcctttgcgcacgacgccccagatgaagtcgccacagaggtcgcaccacgtgtgcgtggcgggccccgcgggctggaagcggtggccacggccagggaccagctgccgtgtggggttgcacgcggtgccccgcgcgatgcgcagcgcgttggcacgctccagccgggtgcggcccttcccagcgcgcccagcgggtgccagctcccgcagctcaatgagctcaggctcccccgacatggcccggttgggcccgtgcttcgctggctttgggcgctagcaagcgcgggccgggcggggccacagggcgggccccgacttcagcgcctcccccaggatccagactgggcggcgggaaggagctgaggagagccgcgcaatggaaacctgggtgcagggactgtggggcccgaaggcggggctgggcgcgctctcgcagagccccccccgccttgcccttccttccctccttcgtcccctcctcacaccccaccccggacggccacaacgacggcgaccgcaaagcaccacgcggagatacccgtgtttctggaggccagctttactgtgctagaggaagagggtccccacatccggcc

SEQ ID NO: 20 - DNA, 1250 bp, Homo sapiens
  source 1..1250: mol_type = genomic DNA
acaggccaagtcctgagtctccagcgaatcaagcaattaaccagtcacttggcctttttgcagaaatccacaaaacgtgtagactcgcttaattccatctcctcctggcctcttcacacggaaattggctgctcgggcgaatgttgtttttctccgcagccatgtgtcgaagggcgattaacattcgctcgacaaaaccctgtcccgggccatttgggagttgcacgcgttcacacccggggctcagcccggtgcgccttcacgcgcagcctttttgtttatgcggctagcagggagaggggcggagaggcagcgaacctctcaatgtgcagcaggccgcgagggctatataaagctcgggtctcggacctactggaactggtcaagattgcccgcgagctggcagcggccttccgcccagctctgcggcgtcatggagaacgcgggaggcgcagagggtacagagagtggagctgcggcgtgcgcggccaccgacggccctacgcggcgcgcgggcgcggactccgggtaggcttccccgcgctgctctgcgcctcccagcttcccccgtctctccctcctgcgcctccccagctcctcccctccaactctccatcccccactgcccggtgccagcgctcggcttcggtggattctcccgggtctgggctcacgtctcctctgcgcgccttgtgctcccctatcccgcagatacgcaggcttctggagaccctgggtggacgccggaggcaagaaagaagaggagacgccgaaccacgccgcggaggcggtgagtgagccacgcgtctcaggccgcgcctcccacgggtgcagaagatcgaccagggctctcgggacgcagggcctgagagagacatgcgagacaccgggtcctgcctctcgcttaacatcccgcctgccggtggccataaaggtgccgacgcggcggatgtgtcctctggagcgatgggccacaggcacccagccgggagcgaggctgctgagagccctggggttacattcccagcgggcacgaggaacactgggtcgtgcacgtttccactttctagaaggaggtgggtgataactggattcacttcctttcctctccagatgcccgatggccctggaatgaccgcagcctcaggaaagctttaccaattcaggcacccagtcaggtgagtgacaggcctcgccgaaggtctcccgctcctccagccccagggaggagccaggggcatcgcgcagcc

SEQ ID NO: 21 - DNA, 1500 bp, Homo sapiens
  source 1..1500: mol_type = genomic DNA
tttcgcctcaataaaactctgacaccaccctccagtcataattagtcacttcttcctctgtacgttggaaacaccgctttatcgtggagactttaaggatatccaagtagagagaattcttccgtgcaagttttgcatcctctcaccactgcctgcgttacccgagttgtaaagggcggctccctgtgtctgccccgctgcaccgatacaccgagctgcgcacggtgcccagcgcagggagaacaaatgatcatctgtccaacgcgcccatttacaggtgaggaaactaaggctccaactcaatcgacgcactctgcccttttgattaccagaaaagtagcaggacaggtgtcctgtcccgccctaccccggcccactaagccggcaccccggctccgacccccggctgtgcccggcgccgccgcggtgcccggcgccgccgcctcgcccggcggggccgcccggagcgcccgcacctccgcccgcttccacctggccgggcccgccccgcccggactcgggactgggaagtgcggcgactcccggaaccagccattggcgccagcgcggggagctgggggtgcagagctgcgggcgcggcgggccacgcaggcggcccccacccccggcctggcctggtctggtctggtctgcgctgccgcgcgggggcgccccctcccaggcccggcgcccgccagccccgctccgccaggtgcagcgcagcgcaggggtgggcgggggtggggctcggcgcgcacgttcacggggcggggagggggcgggtcaggggcgggaccacagccggctgggccggggttctatgcgcatctccggggaggggcggggcgggggcggggccggggcggggcccggtcggtgcactccagacggcgggccgccccctcttcccgccttcctactaccggcccaggattagcgccctgggagcgcgcgccccgctgcctcgccgccacactttcctgggagcggcggccacggaggcaccatgaagaagtcttactcaggtgggcttcgcgcccggggtggggaggggtcggtgtcccgggaccagcgctgctcacctgagtgcctgcggccgggagtggcgaggcgcccccggagctgagcgagtccccgcggcgggcacactgcaggtcgagttcctcccaggacagggccgctgtcgggccgctttcgacctgagccgaccgtcccctgcgctgtctccagcccttgctcgagtgtcggaggggctgccctgggggacgctccctcttcctcgccccttgcaccctcgcaggaatcgctgactttccaggtcggccgggtgctttgggtccctgtgcgtctgtgtgggtgaatggggtcggggctaggtggaggggtgtccttgggttcagcctctagggctggtggtccaggccgcagcatcctttcttcggattctcttcggtttctcctctacttagtggggcacgggacggcctccagatgggaccg

SEQ ID NO: 22 - DNA, 25 bp, synthetic construct
  source 1..25: mol_type = other DNA
tttgatggcggagttacgttcgtcg

SEQ ID NO: 23 - DNA, 21 bp, synthetic construct
  source 1..21: mol_type = other DNA
gtagcggagtgggttaggttc

SEQ ID NO: 24 - DNA, 24 bp, synthetic construct
  source 1..24: mol_type = other DNA
ccgaarctctaaaccaaaacgcaa

SEQ ID NO: 25 - DNA, 21 bp, synthetic construct
  source 1..21: mol_type = other DNA
tcgcgttggttcgagggytcg

SEQ ID NO: 26 - DNA, 21 bp, synthetic construct
  source 1..21: mol_type = other DNA
aagagtagytcggacgttcgg

SEQ ID NO: 27 - DNA, 22 bp, synthetic construct
  source 1..22: mol_type = other DNA
gacgaatcgataccgccgaatc

SEQ ID NO: 28 - DNA, 26 bp, synthetic construct
  source 1..26: mol_type = other DNA
ttatggcgatacgaattcggttggga

SEQ ID NO: 29 - DNA, 19 bp, synthetic construct
  source 1..19: mol_type = other DNA
cggacgttagcgtttgttc

SEQ ID NO: 30 - DNA, 21 bp, synthetic construct
  source 1..21: mol_type = other DNA
cgacaatacgcgcctaacaat

SEQ ID NO: 31 - DNA, 23 bp, synthetic construct
  source 1..23: mol_type = other DNA
cgtcgtcgcgtttgtcgttgaag

SEQ ID NO: 32 - DNA, 18 bp, synthetic construct
  source 1..18: mol_type = other DNA
tcgtcgtggtttcggagc

SEQ ID NO: 33 - DNA, 26 bp, synthetic construct
  source 1..26: mol_type = other DNA
caccaaactccacataaaaacgcgca

SEQ ID NO: 34 - DNA, 20 bp, synthetic construct
  source 1..20: mol_type = other DNA
acgycgggattgcgcgacgc

SEQ ID NO: 35 - DNA, 18 bp, synthetic construct
  source 1..18: mol_type = other DNA
gcgttgttggtcgtggtg

SEQ ID NO: 36 - DNA, 19 bp, synthetic construct
  source 1..19: mol_type = other DNA
grcccaacccgaacgacga

SEQ ID NO: 37 - DNA, 22 bp, synthetic construct
  source 1..22: mol_type = other DNA
tgcgtagagcgagtcgagttcg

SEQ ID NO: 38 - DNA, 24 bp, synthetic construct
  source 1..24: mol_type = other DNA
cgtttttgttagtagcgagtaggc

SEQ ID NO: 39 - DNA, 19 bp, synthetic construct
  source 1..19: mol_type = other DNA
cgcgtccccgcgatcgcgt

SEQ ID NO: 40 - DNA, 23 bp, synthetic construct
  source 1..23: mol_type = other DNA
atcgcgggaaggaacgtatttcg

SEQ ID NO: 41 - DNA, 19 bp, synthetic construct
  source 1..19: mol_type = other DNA
cgagtagggagcgatttcg

SEQ ID NO: 42 - DNA, 17 bp, synthetic construct
  source 1..17: mol_type = other DNA
tcctaaacgcgcatcga

SEQ ID NO: 43 - DNA, 24 bp, synthetic construct
  source 1..24: mol_type = other DNA
tcgaaaacgaaacgaacgcgcgca

SEQ ID NO: 44 - DNA, 24 bp, synthetic construct
  source 1..24: mol_type = other DNA
tttcggcgttcgaggytattacgg

SEQ ID NO: 45 - DNA, 26 bp, synthetic construct
  source 1..26: mol_type = other DNA
cttctactaacarctctactcgacga

SEQ ID NO: 46 - DNA, 24 bp, synthetic construct
  source 1..24: mol_type = other DNA
ttgagttcgcggaacgagttacgg

SEQ ID NO: 47 - DNA, 20 bp, synthetic construct
  source 1..20: mol_type = other DNA
cgggtcgtttgttacgcgtt

SEQ ID NO: 48 - DNA, 23 bp, synthetic construct
  source 1..23: mol_type = other DNA
aaccgcgactctcarctctarcc

SEQ ID NO: 49 - DNA, 24 bp, synthetic construct
  source 1..24: mol_type = other DNA
tcgtcgtcgagtagyttttcgtcg

SEQ ID NO: 50 - DNA, 24 bp, synthetic construct
  source 1..24: mol_type = other DNA
ggaagatattttgaggcgtttcgt

SEQ ID NO: 51 - DNA, 25 bp, synthetic construct
  source 1..25: mol_type = other DNA
cgaactaatcgataactcgaacgcc

SEQ ID NO: 52 - DNA, 24 bp, synthetic construct
  source 1..24: mol_type = other DNA
aagaacgcggcgaagagtttacgg

SEQ ID NO: 53 - DNA, 17 bp, synthetic construct
  source 1..17: mol_type = other DNA
gcggcgtaagaggagga

SEQ ID NO: 54 - DNA, 19 bp, synthetic construct
  source 1..19: mol_type = other DNA
caacgctccgaaacacgac

SEQ ID NO: 55 - DNA, 24 bp, synthetic construct
  source 1..24: mol_type = other DNA
tttcgaggatttggtagcgcgttt

SEQ ID NO: 56 - DNA, 19 bp, synthetic construct
  source 1..19: mol_type = other DNA
gtagggatttgagggtatc

SEQ ID NO: 57 - DNA, 22 bp, synthetic construct
  source 1..22: mol_type = other DNA
cctactcgccgcgctaaatacc

SEQ ID NO: 58 - DNA, 23 bp, synthetic construct
  source 1..23: mol_type = other DNA
tcgcgtacgtaggagcgtagtgg

SEQ ID NO: 59 - DNA, 20 bp, synthetic construct
  source 1..20: mol_type = other DNA
cgcggtagaggtttagtatg

SEQ ID NO: 60 - DNA, 23 bp, synthetic construct
  source 1..23: mol_type = other DNA
actccaaacgaatactacgacga

SEQ ID NO: 61 - DNA, 21 bp, synthetic construct
  source 1..21: mol_type = other DNA
ccgrcccgcgcttactaacgc

SEQ ID NO: 62 - DNA, 19 bp, synthetic construct
  source 1..19: mol_type = other DNA
tcggttgggytcgtgtttc

SEQ ID NO: 63 - DNA, 21 bp, synthetic construct
  source 1..21: mol_type = other DNA
atcgaarcccgccctatarcc

SEQ ID NO: 64 - DNA, 25 bp, synthetic construct
  source 1..25: mol_type = other DNA
tgtatcgaagtcgagtgttcggagg

SEQ ID NO: 65 - DNA, 18 bp, synthetic construct
  source 1..18: mol_type = other DNA
gaaaggcgtttcgggagc

SEQ ID NO: 66 - DNA, 22 bp, synthetic construct
  source 1..22: mol_type = other DNA
ccccttccgaacgacaaatccc

SEQ ID NO: 67 - DNA, 23 bp, synthetic construct
  source 1..23: mol_type = other DNA
tcgcgtaggtagcgtcgagttag

SEQ ID NO: 68 - DNA, 18 bp, synthetic construct
  source 1..18: mol_type = other DNA
agcggcgtagtgatcgtc

SEQ ID NO: 69 - DNA, 24 bp, synthetic construct
  source 1..24: mol_type = other DNA
cgacttcgataaacaacctttcga

SEQ ID NO: 70 - DNA, 22 bp, synthetic construct
  source 1..22: mol_type = other DNA
ttgcggcgttatggagaacgcg

SEQ ID NO: 71 - DNA, 16 bp, synthetic construct
  source 1..16: mol_type = other DNA
ttcgcgagytggtagc

SEQ ID NO: 72 - DNA, 22 bp, synthetic construct
  source 1..22: mol_type = other DNA
cactctctataccctctacgcc

SEQ ID NO: 73 - DNA, 23 bp, synthetic construct
  source 1..23: mol_type = other DNA
acggtcggtttaggtcgaaagcg

SEQ ID NO: 74 - DNA, 22 bp, synthetic construct
  source 1..22: mol_type = other DNA
tcgagtaagggttggagatagc

SEQ ID NO: 75 - DNA, 24 bp, synthetic construct
  source 1..24: mol_type = other DNA
ccaaaacaaarccgctatcgarcc

SEQ ID NO: 76 - DNA, 24 bp, synthetic construct
  source 1..24: mol_type = other DNA
cgtcgtttaggtgacgcgtagtcg

SEQ ID NO: 77 - DNA, 23 bp, synthetic construct
  source 1..23: mol_type = other DNA
agtggtatttaaattttcggcgt

SEQ ID NO: 78 - DNA, 17 bp, synthetic construct
  source 1..17: mol_type = other DNA
cgcaacarctaccgccg

SEQ ID NO: 79 - DNA, 21 bp, synthetic construct
  source 1..21: mol_type = other DNA
tttcgttcgaggcggacgcgt

SEQ ID NO: 80 - DNA, 23 bp, synthetic construct
  source 1..23: mol_type = other DNA
cggattatatgtacggttcgttc

SEQ ID NO: 81 - DNA, 26 bp, synthetic construct
  source 1..26: mol_type = other DNA
cctctttatacttactaccgacgaac

SEQ ID NO: 82 - DNA, 20 bp, synthetic construct
  source 1..20: mol_type = other DNA
agcgacggagtcggcgaaag

SEQ ID NO: 83 - DNA, 21 bp, synthetic construct
  source 1..21: mol_type = other DNA
acgtagtcggyttaagagcga

SEQ ID NO: 84 - DNA, 21 bp, synthetic construct
  source 1..21: mol_type = other DNA
gcgcaaaacgtccaataccga

SEQ ID NO: 85 - DNA, 20 bp, synthetic construct
  source 1..20: mol_type = other DNA
actcgccaatcgccgctccg

SEQ ID NO: 86 - DNA, 20 bp, synthetic construct
  source 1..20: mol_type = other DNA
ggggttaggggytttcgagg

SEQ ID NO: 87 - DNA, 18 bp, synthetic construct
  source 1..18: mol_type = other DNA
ggggttcgtaaggycggg

SEQ ID NO: 88 - DNA, 23 bp, synthetic construct
  source 1..23: mol_type = other DNA
cgaacttcaataccttrcgcgct

SEQ ID NO: 89 - DNA, 22 bp, synthetic construct
  source 1..22: mol_type = other DNA
gtaggtttgggggtagaggaag

SEQ ID NO: 90 - DNA, 23 bp, synthetic construct
  source 1..23: mol_type = other DNA
aatcccaaatacccrctacttcc

SEQ ID NO: 91 - DNA, 26 bp, synthetic construct
  source 1..26: mol_type = other DNA
gtagtgatggattatygttttagtgg

SEQ ID NO: 92 - DNA, 22 bp, synthetic construct
  source 1..22: mol_type = other DNA
cttcrctccaacactccaaatc

SEQ ID NO: 93 - DNA, 22 bp, synthetic construct
  source 1..22: mol_type = other DNA
ttagggattagagaggggaggt

SEQ ID NO: 94 - DNA, 22 bp, synthetic construct
  source 1..22: mol_type = other DNA
ctcarctctarccaatcacctc

SEQ ID NO: 95 - DNA, 26 bp, synthetic construct
  source 1..26: mol_type = other DNA
tgttgagggtgttattggaagatatt

SEQ ID NO: 96 - DNA, 28 bp, synthetic construct
  source 1..28: mol_type = other DNA
ccaataataaaaactacaacctacraac

SEQ ID NO: 97 - DNA, 20 bp, synthetic construct
  source 1..20: mol_type = other DNA
gtagaggyggaagagggagg

SEQ ID NO: 98 - DNA, 23 bp, synthetic construct
  source 1..23: mol_type = other DNA
crctacttactcaataccaactc

SEQ ID NO: 99 - DNA, 25 bp, synthetic construct
  source 1..25: mol_type = other DNA
gggtttyggatttattggaattggt

SEQ ID NO: 100 - DNA, 22 bp, synthetic construct
  source 1..22: mol_type = other DNA
ccrcarctccactctctatacc

SEQ ID NO: 101 - DNA, 17 bp, synthetic construct
  source 1..17: mol_type = other DNA
acctccgcctcccgctc

SEQ ID NO: 102 - DNA, 20 bp, synthetic construct
  source 1..20: mol_type = other DNA
agyttcgggtacggcgcggg

SEQ ID NO: 103 - DNA, 20 bp, synthetic construct
  source 1..20: mol_type = other DNA
ttgtcgaggggyttcgagga

SEQ ID NO: 104 - DNA, 22 bp, synthetic construct
  source 1..22: mol_type = other DNA
gctcttaarccgactacgtccc

SEQ ID NO: 105 - DNA, 20 bp, synthetic construct
  source 1..20: mol_type = other DNA
gtggaggtgggggtattgtt

SEQ ID NO: 106 - DNA, 24 bp, synthetic construct
  source 1..24: mol_type = other DNA
ccaacaarcccaacracataataa

SEQ ID NO: 107 - DNA, 23 bp, synthetic construct
  source 1..23: mol_type = other DNA
gtygttgttgtaggtgtttttgg

SEQ ID NO: 108 - DNA, 20 bp, synthetic construct
  source 1..20: mol_type = other DNA
ccraaaacaccaaactccac

SEQ ID NO: 109 - DNA, 27 bp, synthetic construct
  source 1..27: mol_type = other DNA
gagttggtgtttaaagtagaatttgtt

SEQ ID NO: 110 - DNA, 23 bp, synthetic construct
  source 1..23: mol_type = other DNA
caatacrtcctaccaaaacctcc

SEQ ID NO: 111 - DNA, 25 bp, synthetic construct
  source 1..25: mol_type = other DNA
gaggytattaygggtagtagatttt

SEQ ID NO: 112 - DNA, 22 bp, synthetic construct
  source 1..22: mol_type = other DNA
ctcttacaccctacraacaaca

SEQ ID NO: 113 - DNA, 20 bp, synthetic construct
  source 1..20: mol_type = other DNA
gggagytgggtaaggttttt

SEQ ID NO: 114 - DNA, 22 bp, synthetic construct
  source 1..22: mol_type = other DNA
ttccaacccccraaactctacc

SEQ ID NO: 115 - DNA, 22 bp, synthetic construct
  source 1..22: mol_type = other DNA
ttgtaggggagtgggtgttaag

SEQ ID NO: 116 - DNA, 23 bp, synthetic construct
  source 1..23: mol_type = other DNA
aaactccctctccrcaacaccca

SEQ ID NO: 117 - DNA, 24 bp, synthetic construct
  source 1..24: mol_type = other DNA
tgtaggttgyggatgatgtttgtt

SEQ ID NO: 118 - DNA, 21 bp, synthetic construct
  source 1..21: mol_type = other DNA
cccactacctacccttccttc

SEQ ID NO: 119 - DNA, 24 bp, synthetic construct
  source 1..24: mol_type = other DNA
ccaatcacctcaaaaaaccccgcc

SEQ ID NO: 120 - DNA, 22 bp, synthetic construct
  source 1..22: mol_type = other DNA
cacgacgcacaaatacgccaaa

(19) United States
(12) Patent Application Publication
(10) Pub. No.: US 2025/0066836 A1
(43) Pub. Date: Feb. 27, 2025

(54) METHODS FOR EVALUATING THE METHYLATION STATUS OF A POLYNUCLEOTIDE

(71) Applicant: APR Biosciences Inc., Crown Point, IN (US)

(72) Inventors: Wadiha Freije, Forest Park, IL (US); Igor Brikun, Forest Park, IL (US)

(73) Assignee: APR Biosciences Inc., Crown Point, IN (US)

(21) Appl. No.: 18/722,229

(22) PCT Filed: Dec. 22, 2022
(86) PCT No.: PCT/US2022/082264

§ 371 Date: Jun. 20, 2024

Related U.S. Application Data
(60) Provisional application No. 63/292,852, filed on Dec. 22, 2021.

(51) Int. Cl. C12Q 1/6806

(52) U.S. Cl. CPC C12Q 1/6806; C12Y 201/01

(57) ABSTRACT

Provided are a method for analyzing the methylation status of at least one target sequence in a sample including providing a sample comprising DNA, wherein the DNA comprises at least one target sequence; contacting the sample with at least one methyltransferase that methylates non-cytosine nucleotides; and assaying the sample for the methylation status of one or more cytosine nucleotides in the at least one target sequence, and other related methods.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 128,779 bytes XML file named “SequenceListing_766398,” created Dec. 19, 2022.

### BACKGROUND OF THE INVENTION

Phosphate linked cytosine-guanine (CpG) dinucleotides are statistically underrepresented in the human genome. When they are present, CpG dinucleotides tend to be located within repetitive sequences characterized by low levels of gene expression. Such CpG dinucleotides also tend to feature a methylated cytosine residue.

CpG islands, on the other hand, are genomic sequences with a high density of CpG dinucleotides relative to the rest of the genome. CpG islands include statistical clusters of CpG dinucleotides. While some CpG islands are associated with the promoter region or 5′ end of coding sequences, others are located in introns or genomic regions not known to be associated with coding sequences. CpG islands may be methylated or unmethylated in normal tissues. The methylation pattern of CpG islands may control the expression of tissue specific genes and imprinted genes. Methylation of CpG islands within a gene's promoter regions has been associated with downregulation or silencing of the associated gene. CpG islands may be methylated to varying densities within the same tissue.

Aberrant methylation of cytosines within CpG islands may be a primary epigenetic event that acts to suppress the expression of genes involved in critical cellular processes leading to various diseases such as cancer (Ehrlich, Epigenetics 14, 1141-1163 (2019)). For example, hypermethylation of CpG islands has been detected in tumors and affects genes involved in a variety of cellular processes such as DNA damage repair, hormone response, cell-cycle control, and tumor-cell adhesion/metastasis, leading to tumor initiation, progression, and metastasis (Baylin & Jones, Nat. Rev. Cancer 11, 726-734 (2011); Luo et al., Science 361, 1336-1340 (2018). Aberrant methylation of CpG islands may also be a secondary epigenetic event or a symptom of an upstream abnormality that is the primary event leading to cancer.

It has been proposed that a unique profile of promoter methylation exists for each human cancer, wherein some methylation characteristics are shared and others are cancer-type specific (Esteller et al., Cancer Res. 61, 3225-3229 (2001); Hao et al., Proc. Natl. Acad. Sci. U.S.A. 114, 7414-7419 (2017); Liu et al., Ann. Oncol. 31, 745-759 (2020)). Given that aberrant methylation represents new information not normally present in genomic DNA and that aberrant methylation is a common DNA modification and affects a large number of genomic targets, the detection of disease-specific methylation can be used for diagnostic, predictive and prognostic clinical tests by assaying for the methylation status of at least one target CpG within at least one target sequence. Such tests may be based on CpGs that are aberrantly hypermethylated or hypomethylated in diseased tissues. They may also be based on changes in methylation density in CpG islands. Individual target CpGs or CpG dinucleotide clusters can correlate with a risk for a disease, the presence of a disease, the particular type of a disease or the prognosis of a disease, as well as predicting outcome or the response profile to a treatment regimen.

Noninvasive diagnostics tests have great clinical utility and value (Adashek et al., Cancers 13, 3600 (2021)). They can be performed using liquid biopsies where a small amount of a bodily fluid is analyzed for the presence of disease-specific molecular markers. Liquid biopsies can yield limited amounts of circulating nucleic acids such as cell-free DNA (cf-DNA) and cell-free RNA (cf-RNA) or extracellular vesicles which contain nucleic acids (Eguchi et al., J. Hepathol. 70, 1292-1294 (2019); Hur et al., Cancers 13, 3827 (2021)). The nucleic acids recovered from bodily fluids are derived from both normal and diseased tissues (Mouliere & Rosenfeld, Proc. Natl. Acad. Sci. U.S.A. 112, 3178-3179 (2015); Thierry et al., Cancer Metastasis Rev. 35, 347-376 (2016)). As a result, the nucleic acid present in a sample can contain an unknown mix of methylated and unmethylated target sequences, e.g., markers. Markers are generally present in equal copy numbers in genomic DNA recovered from tissues or cell lines which can be estimated based on the amount of DNA. The proportion of targets of interest in cf-DNA or cf-RNA varies between individuals and within the same individual from day to day. Generally, the copy number of target sequences in cf-DNA and cf-RNA ranges from as little as zero copies to several hundred per nanogram.

Technical challenges that limit the use of methylation markers for liquid biopsies include the small amount of target nucleic acids that can be recovered from circulation and the high sampling error inherent to the analysis of a small sample of a bodily fluid. Combining the molecular information of multiple markers can be helpful in overcoming the high sampling error of liquid biopsies because it increases the likelihood that at least one of the disease-specific methylated targets is present in the liquid biopsy sample. Accurate and sensitive analytical methods to analyze one or more methylation markers from limited amounts of DNA are important for implementing liquid biopsies.

Deamination of unmethylated DNA using bisulfite salts remains an accurate method for determining the methylation status of CpG dinucleotides (Hayatsu, Proc. Jpn. Acad. Ser. B Phys. Biol. Sci. 84, 321-330 (2008)). Bisulfite salts convert unmethylated cytosines to uracils at a faster rate than methylated cytosines resulting in modification to the genomic sequence that enables the detection and quantitation of methylated targets.

However, bisulfite conversion is known to degrade up to 95% of DNA (Genereux et al., Nucleic Acids Res. 36, e150-e150 (2008)). Its application to circulating nucleic acids is challenging because the cf-DNA and cf-RNA recovered from a sample of bodily fluids are available in limited quantities. Liquid biopsies normally yield few nanograms to tens of nanograms, an amount that is difficult to analyze with bisulfite conversion. Accordingly, due to the limited quantities of nucleic acid available for assay in a sample of bodily fluids, it is difficult to simultaneously and accurately assay for the methylation status of multiple markers which is reflected in the paucity of DNA methylation-based diagnostic tests. Two such tests for colon cancer screening are EPI PROCOLON™ (Epigenomics Inc. San Diego, CA, USA) and COLOGUARD™ (Exact Sciences Corp, Madison, WI USA). EPI PROCOLON™ is based on the detection of methylation of a single marker (SEPTIN9) in plasma DNA (Johnson et al. PloS One 9(6): e98238 (2014)). COLOGUARD™ is based on the detection in fecal samples of multiple analytes that include two methylated markers (NDRG4 and BMP3) (Imperiale et al., N. Engl. J. Med. 370(14): 1287-1297 (2014)). Other cancer screening tests that are under development or commercially available include the GALLERI™ test (Grail Inc., Melno Park, Ca, USA) analyze hundreds of thousands of CpG dinucleotides from circulating DNA (Klein et al., Ann. Oncol. 32(9): 1167-1177 (2021); Liu et al., Ann. Oncol. 29(6): 1445-1453 (2018), Ann. Oncol. 31(6): 745-759 (2020)). Galleri is based on targeted bisulfite sequencing of plasma DNA. Despite interrogating the methylation status of over a million CpG dinucleotides, it failed to reach the diagnostic accuracy needed for early cancer screening. Diagnostic tests that are based on too few or too many methylated markers have been challenging to develop and implement. Using too few markers can't overcome the sampling errors of liquid biopsies and disease heterogeneity. The inclusion of too many markers most often leads to a large number of analytical errors (i.e. reduced specificity) and likely detects the normal fluctuations in DNA methylation reducing the accuracy of the test.

Liquid biopsies to screen for, diagnose or monitor complex diseases will likely require the accurate determination of the methylation status of more than 2 markers, more than 10 markers and possibly more than 100 markers. Brikun et al. analyzed the methylation status of 24 to 36 markers for the detection of prostate cancer in biopsy and urine DNA (Brikun et al., Biomark. Res. 2(1): 25 (2014), Clin Epigenetics 10(1): 91 (2018), Exp. Hematol. Oncol. 8(1): 13 (2019)). The analysis of 36 markers from the limited amounts of DNA recovered from urine and formalin-fixed paraffin-embedded (FFPE) biopsy tissues required multiple different bisulfite conditions and the careful selection of markers suitable for analysis under those conditions. Such an approach is difficult to implement for clinical applications where the choice of markers and amount of DNA are limiting.

Thus, methods to enable the accurate simultaneous analysis of a large number of markers from limited quantities of nucleic acids are desirable.

### BRIEF SUMMARY OF THE INVENTION

In an embodiment, the invention provides a method for analyzing the methylation status of at least one target sequence in a sample comprising:
a) providing a sample comprising DNA, wherein the DNA comprises at least one target sequence;
b) optionally, purifying the DNA from the sample to thereby produce purified DNA;
c) optionally, ligating a linker to the DNA to thereby produce tagged DNA;
d) contacting the sample or purified DNA or tagged DNA with at least one methyltransferase that methylates non-cytosine nucleotides; and
e) optionally, contacting the sample or purified DNA or tagged DNA with at least one methyltransferase that methylates cytosine nucleotides; and
f) assaying the sample or purified DNA or tagged DNA for the methylation status of one or more cytosine nucleotides in the at least one target sequence.

In another embodiment, the invention provides a method for analyzing the methylation status of at least one target sequence present in a sample comprising:
a) providing a sample comprising DNA, wherein the DNA comprises at least one target sequence;
b) optionally, purifying the DNA from the sample to thereby produce purified DNA;
c) optionally, ligating a linker to the DNA to thereby produce tagged DNA;
d) contacting the sample or purified DNA or tagged DNA with at least one methyltransferase, wherein the at least one methyltransferase methylates at least 0.7% of cytosine nucleotides in the sample; and
e) assaying the sample or purified DNA or tagged DNA for the methylation status of one or more cytosine nucleotides in the at least one target sequence.

In a further embodiment, the invention provides a method for analyzing the methylation status of at least one target sequence present in a sample comprising:
a) providing a sample comprising RNA, wherein the RNA comprises at least one target sequence;
b) optionally, purifying the RNA from the sample to thereby produce purified RNA;
c) optionally, ligating a linker to the RNA to thereby produce tagged RNA;
d) contacting the sample or purified RNA or tagged RNA with at least one methyltransferase that methylates non-cytosine nucleotides; and
e) assaying the sample or purified RNA or tagged RNA for the methylation status of one or more cytosine nucleotides in the at least one target sequence.

### DETAILED DESCRIPTION OF THE INVENTION

In certain embodiments, the invention relates to methods for assaying the methylation status of a polynucleotide. Generally, the methods of the invention are directed to analyzing the methylation status of at least one target nucleotide in at least one target sequence. In some embodiments, the target sequence is contained within a sample. Optionally, the at least one polynucleotide is purified from the sample, thereby generating at least one purified polynucleotide. Optionally, the at least one polynucleotide is ligated to a linker, thereby generating at least one tagged polynucleotide. In some embodiments, the sample, the purified polynucleotide, or tagged polynucleotide is treated with at least one methyltransferase that methylates non-cytosine nucleotides. In some embodiments, the purified polynucleotide, or tagged polynucleotide is treated with at least one methyltransferase that methylates cytosine nucleotides. In further embodiments, the sample, the purified polynucleotide, or tagged polynucleotide is treated with at least one methyltransferase that methylates non-cytosine nucleotides and at least one methyltransferase that methylates cytosine nucleotides. The method further includes assaying for the methylation status of one or more cytosine nucleotides in the at least one target sequence within the at least one purified polynucleotide, or tagged polynucleotide. In certain embodiments, the polynucleotide is deoxyribonucleic acid (DNA) or ribonucleic acid (RNA).

Thus, an embodiment of the invention includes a method for analyzing the methylation status of a target sequence in a sample for detecting the presence of the target sequence. The method includes providing a sample that includes DNA, optionally purifying the DNA to produce purified DNA, and optionally tagging the DNA to produce tagged DNA. In some embodiments, the sample, purified DNA, or tagged DNA is treated with at least one methyltransferase that methylates non-cytosine nucleotides. In some embodiments, the sample, purified DNA, or tagged DNA is treated with at least one methyltransferase that methylates cytosine nucleotides. In further embodiments, the sample, the purified DNA, or tagged DNA is treated with at least one methyltransferase that methylates non-cytosine nucleotides and at least one methyltransferase that methylates cytosine nucleotides. The method subsequently includes assaying for the methylation status of one or more cytosine nucleotides in the at least one target sequence in the sample, purified DNA, or tagged DNA. The presence or absence of methylation at the one or more cytosine nucleotides indicates the presence or absence, respectively, of methylation at the corresponding cytosine nucleotide in the sample.

Another embodiment of the invention provides for improving the sensitivity and/or specificity of the detection of the methylation status of one or more cytosine nucleotides in an at least one target sequence present in a sample. The method includes providing a sample that includes DNA, optionally purifying the DNA to produce purified DNA, and optionally tagging the DNA to produce tagged DNA. The sample, purified DNA, or tagged DNA is treated with at least one methyltransferase that methylates non-cytosine nucleotides, and/or with at least one methyltransferase that methylates cytosine nucleotides. The method subsequently includes assaying for the methylation status of one or more cytosine nucleotides in the at least one target sequence in the sample, purified DNA, or tagged DNA, wherein the sensitivity and/or specificity of the detection of the methylation status of one or more cytosine nucleotides in the at least one target sequence present in the sample is improved in comparison to a sample that was not treated with the at least one methyltransferase that methylates non-cytosine nucleotides, and/or the at least one methyltransferase that methylates cytosine nucleotides.

Furthermore, an embodiment of the invention includes a method for analyzing the methylation status of a target sequence in a sample for detecting the presence of the target sequence. The method includes providing a sample that includes DNA, optionally purifying the DNA to produce purified DNA, and optionally tagging the DNA to produce tagged DNA. The sample, purified DNA, or tagged DNA is treated with at least one methyltransferase that methylates cytosine nucleotides. In some embodiments, the at least one methyltransferase methylates at least 0.7%, at least 0.71%, at least 0.72%, at least 0.73%, at least 0.74%, at least 0.75%, at least 0.76%, at least 0.77%, at least 0.78%, at least 0.79%, or at least 0.8%, of cytosine nucleotides in the sample. The method subsequently includes assaying for the methylation status of one or more cytosine nucleotides in the at least one target sequence in the sample, purified DNA, or tagged DNA. The presence or absence of methylation at the one or more cytosine nucleotides indicates the presence or absence, respectively, of methylation at the corresponding cytosine nucleotide in the sample.

Another embodiment of the invention includes a method for quantitating the methylation status of at least one target sequence present in a sample to quantitate the percentage of the at least one target sequence that is methylated. The method includes providing a sample that includes DNA, optionally purifying the DNA to produce purified DNA, and optionally tagging the DNA to produce tagged DNA. The sample, purified DNA, or tagged DNA is treated with one or both of (i) at least one methyltransferase that methylates cytosine nucleotides; and (ii) at least one methyltransferase that methylates non-cytosine nucleotides. In some embodiments, the at least one methyltransferase that methylates cytosine nucleotides methylates at least 0.7%, at least 0.71%, at least 0.72%, at least 0.73%, at least 0.74%, at least 0.75%, at least 0.76%, at least 0.77%, at least 0.78%, at least 0.79%, or at least 0.8%, of cytosine nucleotides in the sample. The method subsequently includes assaying for the methylation status of one or more cytosine nucleotides in the at least one target sequence in the sample, purified DNA, or tagged DNA. The method further includes comparing the amount of methylated and unmethylated cytosine nucleotides in the at least one target sequence to a corresponding amount in a standard. The presence or absence of methylation at the one or more cytosine nucleotides indicates the presence or absence, respectively, of methylation at the corresponding cytosine nucleotide in the sample. When this method is used to assay two or more cytosine nucleotides in the at least one target sequence in the sample, it can be used for analyzing the density of methylation of the at least one target sequence present in a sample to improve the quantitation of the methylation of two or more cytosines within the at least one target sequence.

The invention yet further includes a method for stabilizing at least one polynucleotide in a sample. Optionally, the at least one polynucleotide is purified from the sample, thereby generating at least one purified polynucleotide. Optionally, the at least one polynucleotide is ligated to a linker, thereby generating at least one tagged polynucleotide. The sample, the purified polynucleotide, or tagged polynucleotide is treated with (i) at least one methyltransferase that methylates cytosine nucleotides; and/or (ii) at least one methyltransferase that methylates non-cytosine nucleotides. In some embodiments, when at least one methyltransferase that methylates cytosine nucleotides is used, the at least one methyltransferase methylates at least 0.7%, at least 0.71%, at least 0.72%, at least 0.73%, at least 0.74%, at least 0.75%, at least 0.76%, at least 0.77%, at least 0.78%, at least 0.79%, or at least 0.8%, of cytosine nucleotides in the sample. Following the methyltransferase treatment, the sample, the purified polynucleotide, or tagged polynucleotide has increased stability in comparison to a sample, purified polynucleotide, or tagged polynucleotide that has not been undergone such treatment. Optionally, the method includes assaying for the methylation status of one or more cytosine nucleotides in the at least one target sequence within the at least one purified polynucleotide, amplified polynucleotide, or tagged polynucleotide. In certain embodiments, the polynucleotide is deoxyribonucleic acid (DNA) or ribonucleic acid (RNA).

In the methods described herein, when “at least one methyltransferase” is recited, in certain embodiments, the at least one methyltransferase is at least two, at least three, at least four, at least five, at least six, at least seven, at least eight, at least nine, or at least ten methyltransferases. In some embodiments, the at least one methyltransferase is at least two, at least three, at least four, or at least five methyltransferases, preferably at least two methyltransferases, or at least three methyltransferases. In certain other embodiments, the at least one methyltransferase is one, two, three, four, five, six, seven, eight, nine or ten methyltransferases. In some embodiments, the at least one methyltransferase is one, two, three, four, or five methyltransferases, preferably one, two, three or four methyltransferases, more preferably one, two or three methyltransferases, yet more preferably, one or two methyltransferases, still more preferably one methyltransferase. These apply equally to when the at least one methyltransferase in question methylates adenine, non-cytosine, cytosine nucleotides, or any combination thereof. In certain other embodiments, in which a combination of at least one methyltransferase that methylates adenine or non-cytosine nucleotides and at least one methyltransferase that methylates cytosine nucleotides is used, the combination is any combination, wherein the at least one methyltransferase that methylates adenine or non-cytosine nucleotides is at least two, at least three, at least four, at least five, at least six, at least seven, at least eight, at least nine, or at least ten such methyltransferases, preferably at least one, at least two, at least three or at least four methyltransferases, more preferably at least one, at least two or at least three methyltransferases, yet more preferably, at least one or at least two methyltransferases, still more preferably, at least one methyltransferase, and the at least one methyltransferase that methylates cytosine nucleotides is at least two, at least three, at least four, at least five, at least six, at least seven, at least eight, at least nine, or at least ten such methyltransferases, preferably at least one, at least two, at least three or at least four methyltransferases, more preferably at least one, at least two or at least three methyltransferases, yet more preferably, at least one or at least two methyltransferases, still more preferably, at least one methyltransferase.

The invention described herein further provides a method of preparing a sample for methylation analysis. The sample can be any suitable sample. In certain embodiments the sample contains one or more of the following mammalian bodily fluids: blood, blood plasma, blood serum, urine, sputum, ejaculate, semen, prostatic fluid, tears, sweat, saliva, lymph fluid, bronchial lavage, pleural effusion, peritoneal fluid, meningeal fluid, amniotic fluid, glandular fluid, fine needle aspirates, nipple aspirate fluid, spinal fluid, conjunctival fluid, vaginal fluid, duodenal juice, pancreatic juice, pancreatic ductal epithelium, pancreatic tissue bile, cerebrospinal fluid, or any combination thereof.

The invention provides methods for analyzing the methylation status of one or more target sequences in samples that include polynucleotides, wherein the sample and/or polynucleotides contained therein are contacted with at least one methyltransferase. As used herein, “target sequence” refers to a nucleotide sequence that includes one or more target cytosine nucleotides with a methylation status of interest. For example, in certain embodiments, target sequences include genomic CpG islands, or portions thereof, which contain one or more target cytosines whose methylation status is associated with a disease. In certain embodiments, the disease is a cancer, e.g., leukemia (e.g., lymphoblastic, myeloid, hairy cell), adrenocortical carcinoma, anal cancer, appendix cancer, astrocytoma, basal cell carcinoma, extrahepatic bile duct cancer, bladder cancer, bone cancer, brain cancer, gliomas, breast cancer, bronchial adenomas, carcinoid tumors, cervical cancer, myeloproliferative disorders, colon cancer, endometrial cancer, esophageal cancer, eye cancer, gallbladder cancer, stomach cancer, gastrointestinal tumors, head and neck cancer, liver cancer, lymphomas (e.g., Hodgkin's, Non-Hodgkin's, Burkitt's, T-cell, central nervous system, and AIDS-related), sarcomas, kidney cancer, laryngeal cancer, lip and oral cavity cancer, liver cancer, lung cancer, macroglobulinemia, melanoma, Merkel cell carcinoma, mesothelioma, myeloproliferative disorders, nasal and paranasal sinus cancer, nasopharyngeal cancer, neuroblastoma, ovarian cancer, pancreatic cancer, parathyroid cancer, penile cancer, pharyngeal cancer, pheochromocytoma, pituitary tumor, pancreatic cancer, prostate cancer, rectal cancer, kidney cancer, salivary gland cancer, skin cancer (non-melanoma), testicular cancer, throat cancer, thyroid cancer, trophoblastic tumor ureter and renal pelvis cancer, urethral cancer, uterine cancer, vaginal cancer, or vulvar cancer. In certain embodiments, the disease is lung cancer, prostate cancer, ovarian cancer, colon cancer, liver cancer, pancreatic cancer, thyroid cancer, skin cancer, head and neck cancer, brain cancer, or hematological cancer. In certain embodiments, the disease is prostate cancer. In other certain embodiments, the disease is an infectious, immunological, or neurological disease. The methylation status of a target sequence refers to whether at least one target nucleotide (e.g., a target cytosine in a CpG dinucleotide) is methylated or unmethylated. Alternatively, the methylation status of a target sequence refers to whether multiple (i.e., a plurality of) target nucleotides in a target sequence are each methylated or unmethylated. When referring to multiple or a plurality of target nucleotides, methylation status in some cases refers to the methylation pattern or methylation density of a target sequence.

In certain embodiments, the samples referenced herein include, for instance, RNA and/or DNA. In some embodiments, the DNA is genomic DNA. The samples can be derived from eukaryotes such as any fungi, plants or animals. In some embodiments, an animal sample is derived from a mammal. Non-limiting examples of suitable mammals include human, primates, monkeys, rat, mouse, pig, horse, and cow. In certain embodiments, samples include tissue samples and/or cells, e.g., those acquired from an organism by biopsy, surgical resection, or any other suitable extractive technique. In some embodiments, samples include tissues and cells cultured in vitro.

In certain embodiments, samples include bodily fluids, which, generally, refer to mixtures of macromolecules obtained from an organism. Thus, in some embodiments, samples include blood, blood plasma, blood serum, urine, sputum, ejaculate, semen, tears, sweat, saliva, lymph fluid, bronchial lavage, pleural effusion, peritoneal fluid, meningeal fluid, amniotic fluid, glandular fluid, fine needle aspirates, nipple aspirate fluid, spinal fluid, conjunctival fluid, vaginal fluid, duodenal juice, pancreatic juice, pancreatic ductal epithelium, pancreatic tissue bile, cerebrospinal fluid, or any combination thereof. In some embodiments, samples include solutions or mixtures made from homogenized solid material such as feces. In further embodiments, samples include experimentally/clinically separated fractions from bodily fluids, tissues, and/or cells. Preferably, samples include tissue biopsies, plasma, urine, saliva, bronchial lavage, and fine needle aspirate.

The methods of the invention are suitable for the methylation analysis of nucleic acid-containing samples. In certain embodiments, the nucleic acid is RNA or DNA. In the case of DNA, in some embodiments, genomic DNA is used. Methylation is known to exert a modest effect on the conformation and stability of DNA helices. Methylation of short duplex polynucleotides at the N⁶-amino group of adenine residues may exert a reduction in the stability of DNA helices (Engel & von Hippel, Biochemistry 13, 4143-4158 (1974); J. Biol. Chem. 253, 927-934 (1978). It is difficult to extrapolate from these studies the impact of adenine methylation on the structure and stability of longer, highly heterogenous, complex single stranded polynucleotides. However, without being bound to a particular theory, it is thought to reduce the number of conformations that a DNA fragment can adopt once denatured and rendered single stranded. Cytosine residues are known to react with bisulfite salts, and the kinetics of the reaction are dependent on the methylation status of the cytosine residues. Methylation of cytosines at the C5 position reduces their rate of sulfonation in the presence of bisulfite salts, an observation that led to the development of methods to determine the methylation status of cytosines (Frommer et al., “A genomic sequencing protocol that yields a positive display of 5-methylcytosine residues in individual DNA strands,” Proc. Natl. Acad. Sci. U.S.A., 89(5): 1827-1831 (1992); Hayatsu, “Bisulfite modification of nucleic acids and their constituents,” Prog. Nucleic Acid Res. Mol. Biol., 16: 75-124 (1976); Hayatsu, “Discovery of bisulfite-mediated cytosine conversion to uracil, the key reaction for DNA methylation analysis—a personal account,” Proc. Jpn. Acad. Ser. B Phys. Biol. Sci., 84(8): 321-330 (2008)). Without being bound to theory, the reduction in sulfonation of methylated cytosines within a polynucleotide may reduce its degradation during subsequent desulfonation which converts cytosines into uracils.

The increase in methylation resulting from contacting the sample with at least one methyltransferase is expected to alter the secondary structure of single stranded polynucleotides. It may increase their stability under varying ionic conditions and temperatures and/or alter the rate of reactions with different salts or enzymes. Without being bound to a particular theory, such increased methylation may also render the polynucleotide less susceptible to degradation or alter its rate of degradation during subsequent analysis of the methylation status of the polynucleotide. Furthermore, the presence of methylated cytosines within a polynucleotide improves the deamination of neighboring unmethylated cytosines in the presence of bisulfite salts (Genereux et al., “Errors in the bisulfite conversion of DNA: modulating inappropriate- and failed-conversion frequencies,” Nucleic Acids Res., 36(22): e150-e150 (2008); Grunau et al., “Bisulfite genomic sequencing: systematic investigation of critical experimental parameters,” Nucleic Acids Res., 29(13): E65-65 (2001)). The potential reduction of secondary structure of adenine-methylated single-stranded polynucleotides may further improve their reaction with bisulfite salts as compared with unmethylated single-stranded polynucleotides. The inventors have found that contacting a sample in vitro with at least one methyltransferase can increase the detectability of the methylation of the nucleic acid within the sample during subsequent manipulations. Accordingly, the methods of the invention are particularly useful for the methylation analysis of samples with relatively small amounts of nucleic acid and for applications requiring the analysis of multiple, and in some cases, many, methylated markers.

For example, in some embodiments, the sample includes 500, 100, 75, 50, 40, 30, 20, 19, 18, 17, 16, 15, 14, 13, 12, 11, 10, 9, 8, 7, 6, 5, 4, 3, 2, or 1 nanograms (ng) of DNA or RNA. In further embodiments, the sample includes less than 20 ng of DNA, less than 15 ng of DNA, less than 10 ng of DNA, less than 5 ng, or less than 1 ng of DNA. In yet other embodiments, samples include as little as 500, 400, 300, 200, 100, or less than 10 picograms of DNA. In one embodiment, the sample includes less than 15 ng of DNA. In embodiments wherein the sample contains “less than [a recited amount] of DNA,” the sample contains some amount of DNA, i.e., the sample does not completely lack DNA. Such samples are exemplified by nucleic acids isolated from bodily fluids which usually include relatively small amounts of circulating DNA. In some embodiments, the bodily fluids contain urine and/or plasma or fractionated nucleic acids from urine and/or plasma. In this regard, the amount of circulating DNA recovered from different individuals can vary as much as 10-fold or more. For example, the amount of DNA recovered from bodily fluids can range from less than one to over 15 ng/ml of plasma in healthy individuals and from less than one nanogram to over a microgram per ml from cancer patients (Jahr et al., DNA fragments in the blood plasma of cancer patients: quantitations and evidence for their origin from apoptotic and necrotic cells,” Cancer Res., 61(4): 1659-1665 (2001); Altimari et al., “Diagnostic role of circulating free plasma DNA detection in patients with localized prostate cancer,” Am. J. Cin Pathol., 129(5): 756-762 (2008); Sozzi et al., “Quantification of free circulating DNA as a diagnostic marker in lung cancer,” J. Clin. Oncol., 21(21): 3902-3908 (2003); Diehl et al., “Detection and quantification of mutations in the plasma of patients with colorectal tumors,” Proc. Natl. Acad. Sci. USA, 102(45): 16368-16373 (2005); Diehl et al., “Circulating mutant DNA to assess tumor dynamics,” Nat. Med. 14(9): 985-990 (2008); Diaz et al., “Liquid biopsies: genotyping circulating tumor DNA,” J. Clin. Oncol., 32(6): 579-586 (2014); Cheng et al., “Cell-Free Circulating DNA Integrity Based on Peripheral Blood as a Biomarker for Diagnosis of Cancer: A Systematic Review,” Cancer Epidemiol. Biomarker Prev., 26(11): 1595-1602 (2017); Fleischhacker et al., “Circulating nucleic acids (CNAs) and cancer—a survey,” Biochim. Biophys. Acta, 1775(1): 181-232 (2007); Goebel et al., “Circulating nucleic acids in plasma or serum (CNAPS) as prognostic and predictive markers in patients with solid neoplasias,” Dis. Markers, 21(3): 105-120 (2005)). The amount of DNA recovered from cancer patients is more variable but can be comparable to those observed for many healthy individuals, particularly for patients diagnosed with early-stage disease. Higher amounts of cf-DNA are generally recovered from patients diagnosed with advanced disease but not always. The inventors have routinely recovered DNA amounts ranging from less than 1 nanogram to greater than 50 nanograms of circulating DNA per milliliter of urine or plasma. Although extracting a larger sample can increase the amount of DNA, there is usually a limited volume that can be reasonably obtained and analyzed in a clinical setting. Furthermore, the copy number of various polynucleotides in circulating DNA varies widely within and between individuals yielding nucleic acid samples with unknown and highly heterogeneous composition. The methods of the invention are useful for the analysis of polynucleotides present in heterogenous samples of unknown composition and samples that include otherwise depleted or scarce genomic DNA.

In some embodiments, the sample comprises fragments of fewer than 500, fewer than 450, fewer than 400, fewer than 350, fewer than 300, fewer than 250, fewer than 200, fewer than 150, fewer than 100, or fewer than 50 contiguous nucleotides, wherein the fragments include the at least one target sequence. In an embodiment, the sample comprises fragments of fewer than 150 contiguous nucleotides, wherein the fragments include the at least one target sequence. In certain embodiments, the sample comprises fragments of a range of size of contiguous nucleotides, wherein the lower and upper range of the range is one of the amounts recited in this paragraph, e.g., 50-500, 200-450, and 50-150, 35-75, 35-55 and 20-30 contiguous nucleotides.

As discussed herein, in certain embodiments, the sample polynucleotide, e.g. genomic DNA, is optionally purified. As used herein, “purified” refers to the separation of polynucleotide material from some or most of the tissue, cellular, macromolecular, or other non-polynucleotide material previously associated with the polynucleotide. It can also refer to the separation of the target polynucleotide from other polynucleotides present in the sample. Any suitable purification method known in the art can be used. Genomic DNA can include fragments of differing lengths, including fragments as short as 35 to 200 base pairs (bp) in length to fragments that are over 1 million base pairs (as used herein, “bp” can also refer to the length, in nucleotides, of single stranded polynucleotides).

As discussed herein, in certain embodiments, the sample polynucleotide is optionally ligated to a linker (or adapter, which terms are used herein synonymously) to produce tagged nucleic acid. The linker or adapter can be any suitable linker or adapter known in the art. In some embodiments, the linker or adapter is oligonucleotides made of naturally occurring bases such as ribonucleotides or deoxyribonucleotides. In further embodiments, the linker is made of modified or non-natural bases such as locked or unlocked nucleic acids (LNA) or a combination of natural and unnatural nucleotides (Crouzier et al., “Efficient reverse transcription using locked nucleic acid nucleotides towards the evolution of nuclease resistant RNA aptamers,” P1oS One, 7(4): e35990 (2012); Malyshev et al., “PCR with an expanded genetic alphabet,” J. Am. Chem. Soc., 131(41): 14620-14621 (2009); Soler-Bistué et al., “Bridged Nucleic Acids Reloaded,” Molecules, 24(12): E2297 (2019); Yang et al., “Expanded genetic alphabets in the polymerase chain reaction,” Angew. Chem. Int. Ed Engl., 49(1): 177-180 (2010)). In certain embodiments, the linker or adapter includes unique molecular identifiers such as short random sequences to enable counting of the copy number of the target sequence that is present in the sample (Hong et al., “Incorporation of unique molecular identifiers in TruSeq adapters improves the accuracy of quantitative sequencing,” BioTechniques, 63(5): 221-226 (2017); Kinde et al., “Detection and quantification of rare mutations with massively parallel sequencing,” Proc. Natd. Acad. Sci. U.S.A., 108(23): 9530-9535 (2011); Kivioja et al., “Counting absolute numbers of molecules using unique molecular identifiers,” Nat. Methods, 9(1): 72-74 (2011)). In certain embodiments, the linker is ligated to the DNA after enzymatic treatment to generate ends suitable for ligation such as blunt ends or ends with an overhang. In some embodiments, the linker is ligated to single stranded DNA using ligases such as SplintR ligase or thermostable 5′App DNA/RNA ligase. In further embodiments, the linker is introduced on target polynucleotides by amplification using a library of linkers composed of target specific fragments, and one or more universal primer(s). In some embodiments, the linker optionally includes a unique molecular identifier and a fixed barcode. In some embodiments in which DNA is used, the DNA is optionally ligated to generate non-contiguous larger fragments. In certain embodiments, the linker is introduced to the sample polynucleotide or purified DNA before or after the DNA deamination that occurs during the assay of the sample or purified DNA for the methylation status of one or more cytosine nucleotides in the sample or purified DNA.

The methods of the invention include contacting the sample, purified nucleic acid or tagged nucleic acid with at least one methyltransferase. The at least one methyltransferase can be any suitable methyltransferase. Zhang et al., Virology 240(2): 366-75 (1998); Que et al. Gene 190(2): 237-44 (1997); Xu et al. Nucleic Acids Res. 26(17): 3961-6 (1998); Chan et al. Nucleic Acids Res. 32(21): 6187-6199 (2004); Miura et al. BMC Biotechnol. 22:33 (2022); Coy et al. Front. Microbiol. 11:887 (2020); Clark et al. Nucleic Acids Res. 40(4): e29 (2012); Jensen et al. Nature Comms. 10: 3311 (2019). Suitable methyltransferases include wild-type methyltransferases and those that have been modified in vitro. In some embodiments, the specificity of the in vitro modified methyltransferase is changed as a result of the one or more modifications. Any methyltransferase that methylates one to six nucleotides, or a plurality of such methyltransferases, is suitable for the invention. A plurality of methyltransferases can be tailored to a panel of selected markers based on, for instance, the sequence of the markers and the assay conditions used.

In some embodiments, the at least one methyltransferase includes at least one methyltransferase that methylates adenine nucleotides. In some embodiments, the at least one methyltransferase includes at least one methyltransferase that methylates cytosine nucleotides. In some embodiments, the at least one methyltransferase includes at least one methyltransferase that methylates adenine nucleotides, and at least one methyltransferase that methylates cytosine nucleotides.

In some embodiments, the at least one methyltransferase includes at least one methyltransferase that methylates adenine nucleotides present in the sample and/or at least one target sequence therein. In some embodiments, the at least one methyltransferase includes at least one methyltransferase that methylates adenine nucleotides within a recognition sequence that comprises the sequence GATC, GTAC, TCGA, CATG, AATT, GAWTC, SATC, AACCA, ACATC, GAATTC, GACGTC, CACAG, RAR, AG, or any combination thereof. In further embodiments, the at least one methyltransferase includes at least one methyltransferase that methylates adenine nucleotides that are not within a specific recognition sequence.

In some embodiments, the at least one methyltransferase includes two or more methyltransferases that methylate adenine nucleotides, wherein the recognition sequence for each of the two or more methyltransferases is GATC, GTAC, TCGA, CATG, AATT, GAWTC, SATC, AACCA, ACATC, GAATTC, GACGTC, CACAG, RAR or AG. In some embodiments, when considered together, the two or more methyltransferases methylate adenine residues within one of the following combinations of recognition sequences: AG GATC, AG GTAC, AG TCGA, AG CATG, AG AATT, RAR GATC, RAR GTAC, RAR TCGA, RAR CATG, RAR AATT, GATC GTAC, GATC AATT, GATC TCGA, GTAC AATT, GTAC TCGA, AATT TCGA, GATC GAWTC, GTAC GAWTC, AATT GAWTC, TCGA GAWTC, GTAC SATC, TCGA SATC, and AATT SATC. In other embodiments, one adenine methyltransferase does not require a recognition sequence and a second adenine methyltransferase methylates adenine within one of the following recognition sequences: AATT, GTAC, GATC, CATG, TCGA and GAWTC.

In some embodiments, the at least one methyltransferase is EcoGII, which methylates≥50% of adenine residues within a polynucleotide (New England Biolabs, Ipswich, MA USA). In other embodiments, the at least one methyltransferase that methylates adenine nucleotides is M.EcoKDam, M.CviQI, M.CviQXI, M.CvQII, M.TaqI, M.Tsp509I, M1.Bst19I, M.AatII, M.EcoR1, or any combination thereof. In further embodiments, the at least one methyltransferase includes any two of the following methyltransferases: M.CviQI, M.CviQXI, M.CvQII, M.EcoKDam, M.EcoGII, M.Tsp509I and M.TaqI. In some embodiments, two methyltransferases are employed, wherein the two methyltransferses are M.CviQI M.EcoKDam; M.CviQI M.CviQXI, M.CviQI M.CvQII, M.CviQIM.Tsp509I; M.CviQI M.TaqI; M.CviQXI M.EcoKDam, M.CviQXI M.Tsp5091, M.CviQXI M.TaqI, M.CvQII M.EcoKDam, M.CvQII M.Tsp5091, M.CvQII M.TaqI, M.EcoKDam M.Tsp509I; M.EcoKDam M.TaqI; M.Tsp5091 M.TaqI; M.EcoGII M.CviQI; M.EcoGII M.EcoKDam; M.EcoGII M.Tsp5091; or M.EcoGII M.TaqI. In other embodiments, the at least one methyltransferase includes three, four, five, six or more methyltransferases that methylate adenine nucleotides at a total of three or more of the following recognition sequences: AG, RAR, GATC, GTAC, TCGA, CATG, AATT, GAWTC, SATC, AACCA, ACATC, GAATTC, GACGTC, CACAG. In other embodiments, the three or more adenine methyltransferases, when considered together, methylate adenines within the combination of the following recognition sites: RAR GATC GTAC, RAR GATC TCGA, RAR GATC AATT, RAR TCGA AATT, GATC GTAC TCGA; GATC TCGA AATT; GTAC AATT TCGA; and GATC GTAC TCGA AATT. In other embodiments, the at least one adenine methyltransferase includes three or more of the following methyltransferases: M.CviQ1, M.CviQXI, M.CvQII, M.EcoKDam, M.EcoGII, M.Tsp509I and M.TaqI. In some embodiments, the methylases are M.CviQ1 M.EcoKDam M.Tsp509I; M.CviQ1 M.EcoKDam M.TaqI; M.EcoKDam M.Tsp509I M.TaqI; M.CviQ1 M.EcoKDam M.Tsp509I M.TaqI; M.CviQXI M.EcoKDAM M.Tsp509I, M.CviQXI M.EcoKDAM M.TaqI, M.CviQXI M.EcoKDAM M.Tsp509I M.TaqI, M.CvQII M.EcoKDAM M.Tsp509I, M.CvQII M.EcoKDAM M.TaqI, M.CvQII M.EcoKDAM M.Tsp509I M.TaqI, or M.EcoGII M.CviQ1 M.EcoKDam M.Tsp5091 M.TaqI.

In some embodiments, the at least one methyltransferase includes at least one methyltransferase that methylates cytosine residues within a recognition sequence that comprises the sequence CC, CCD, RGC, CGR, RGCB, AGCT, GGCC, GCGC, GTAC, GATC, TCGA, CCGG, GCNGC, CCWGG, RCATGY, GAGCTC, GC, or any combination thereof. In some embodiments, the at least one methyltransferase includes at least two methyltransferases that methylate cytosine nucleotides, wherein each methyltransferase methylates within a recognition sequence that comprises CC, CCD, RGC, CGR, RGCB, AGCT, GGCC, GCGC, GTAC, GATC, TCGA, CCGG, GCNGC, CCWGG, RCATGY, and GA.

GCTC, GC, or any combination thereof. In some embodiments, the two methyltransferases, when considered together, methylate cytosine residues within the following combinations of recognition sequences: CCD AGCT, CCD GCGC, CCD GTAC, CCD GATC, CCD TCGA, CCD RCATGY, CCD GAGCTC, CGR AGCT, CGR GCGC, CGR GTAC, CGR GATC, CGR TCGA, CGR RCATGY, CGR GAGCTC, RGCB GGCC, RGCB GCGC, RGCB GTAC, RGCB GATC, RGCB TCGA, RGCB CCGG, RGCB GCNGC, RGCB CCWGG, AGCT GGCC, AGCT GCGC, AGCT GTAC, AGCT TCGA, AGCT CCGG, AGCT GCNGC, AGCT CCWGG, AGCT RCATGY, GGCC, CCGG, GGCC GTAC, GGCC GATC, CCGG TCGA, GGCC CCGG, CCGG GTAC, CCGG GATC, CCGG TCGA, and CCGG GGCC. In some embodiments, the at least one methyltransferase that methylates cytosine nucleotides is M.AluI, M.BamHI, M.CviPI, M.CviPII, M.CviQIX, M.CviQVIII, M.CviQx, M.EcoKDcm, M.EsaLHCI, M.EsaBC2I, M.HaeIII, M.HhaI, M.HpaII, M.MspI, M.NspI, M.RsaI, M.Sau3AI, or any combination thereof. In another embodiment, the at least one methyltransferase is M.CviPI, M.CviPII, M.CviQIX, M.CviQVIII, or any combination thereof. In yet another embodiment, the at least one methyltransferase is M.CviPI. In yet another embodiment, the at least one methyltransferase is M.CviQIX. In yet another embodiment, the at least one methyltransferase is M.Alu.

In some embodiments, the at least one cytosine methyltransferase includes two or more of the following methyltransferases, M.Alu, M.HaeIII, M.EcoKDcm, M.HhaI, M.HpaII, M.MspI, M.NspI, M.Sau3AI, M.CviP1, M.CviPII, M.CviQIX, M.CviQVIII, phi3T and Spr methyltransferases. In some embodiments, the cytosine methyltransferases are one of the following combinations (demarcated by semicolon): M.Alu M.HaeIII M.EcoKDcm; M.Alu M.HaeIII M.HhaI; M.Alu M.HaeIII M.HpaII; M.AluI M.HaeIII M.MspI; M.Alu M.HaeIII M.NspI; M.Alu M.HaeIII M.Sau3A; M.Alu M.HhaI M.HpaII; M.Alu M.HhaI M.MspI; M.AluI M.HhaI M.NspI; M.Alu M.HhaI M.Sau3A; M.Alu M.HhaI M.Sau3AI; M.HaeIII M.HhaI M.HpaII; M.HaeIII M.EcoKDcm M.HhaI; M.HaeIII M.EcoKDcm M.HpaII; M.HaeIII M.EcoKDcm M.MspI; M.HaeIII M.EcoKDcm M.NspI; M.HaeIII M.EcoKDcm M.Sau3A; M.HaeIII M.HhaI M.MspI; M.HaeIII M.HhaI M.NspI; M.HaeIII M.HhaI M.Sau3A; M.HaeIII M.HpaII M.MspI; M.HaeII M.HpaII M.NspI; M.HaeIII M.HapII M.Sau3A; M.HhaI M.HpaII M. MspI; M.HhaI M.HpaII M.NspI; M.HhaI M.HpaII M.Sau3AI; M.HhaI M.MspI M.NspI; M.HhaI M.MspI M.Sau3A; M.HpaII M.MspI M.NspI; M.HpaII M.MspI M.Sau3AI; M.MspI M.NspI M.Sau3A; M.Alu M.HaeIII M.HhaI M.HpaII; M.Alu M.HaeIII M.HhaI M.MspI; M.AluI M.HaeIII M.HhaI; M.NspI; M.AluI M.HaeIII M.HhaI M.Sau3A; M.Alu M.HaeIII M.HpaII M.Sau3A; M.AluI M.HhaI; M.HpaII M.Sau3AI; M.HaeIII M.HhaI M.HpaII M.MspI; HaeIII M.HhaI M.HpaII M.NspI; HaeIII M.HhaI M.HpaII M.Sau3A; M.HhaI M.HpaII M.MspI M.NspI; M.HhaI M.HpaII M.MspI M.Sau3A; M.HpaII M.MspI M.NspI M.Sau3A; M.AluI M.HaeIII M.HhaI M.HpaII M.MspI; M.Alu M.HaeIII M.HhaI M.HpaII M.NspI; M.AluI M.HaeIII M.HhaI M.HpaII M.Sau3A; M.HaeIII M.HhaI M.HpaII M.MspI M.NspI; M.HaeIII M.HhaI M.HpaII M.MspI M.Sau3A; M.HaeIII M.HhaI M.HpaII M.MspI M.NspI; M.HaeIII M.HhaI M.HpaII M.MspI M.Sau3A; M.HhaI M.HpaII M.MspI M.NspI M.Sau3A; M.Alu M.HaeIII M.HhaI M.HpaII M.MspI M.NspI; M.Alu M.HaeIII M.HhaI M.HpaII M.NspI M.Sau3A; and M.AluI M.HaeIII M.HhaI M.HpaII M.MspI M.NspI M.Sau3A. In other embodiments, methyltransferases from the phi 3T and Spr phages of Bacillus subtilis are used individually or in combination with one or more other cytosine methyltransferases (Balganesh et al., “Construction and use of chimeric SPR/phi 3T DNA methyltransferases in the definition of sequence recognizing enzyme regions,” EMBO J., 6(11): 3543-3549 (1987); Behrens et al., “Organization of multispecific DNA methyltransferases encoded by temperate Bacillus subtilis phages,” EMBO J., 6(4): 1137-1142 (1987); Wilke et al., “Sequential order of target-recognizing domains in multispecific DNA-methyltransferases,” EMBO J., 7(8): 2601-2609 (1988). In other embodiments, the at least one cytosine methyltransferase is M.CviPI, M.CviPII, M.CviQIX or M.CviQVIII. In still another embodiment, the at least one cytosine methyltransferase is M.CviPI. In still another embodiment, the at least one cytosine methyltransferase is M.CviQIX.

In some embodiments, at least one adenine methyltransferase and at least one cytosine methyltransferase are used, wherein the at least one adenine and the at least one cytosine methyltransferases include two or more of the following methyltransferases: M.CviQI, M.CviQX1, M.CvQII, M.EcoKDam, M.Tsp509I, M.TaqI, M.EcoGII, M.EcoKDcm, M.Alu, M.HaeIII, M.HhaI, M.HpaII, M.MspI, M.NspI, M.Sau3AI, M.CviPI, M.CviPII, M.CviQIX, M.CviQVIII and M.CviQX. In some embodiments, the at least one adenine methyltransferase and at least one cytosine methyltransferase are one of the following combinations (demarcated by semicolon): M.CviQX1 M.CviPI; M.CviQX1 M.CviPII; M.CviQX1 M.CviQIX; M.CviQX1 M.CviQVIII; M.CviQX1 M.CviQX; M.EcoGII M.CviPI; M.EcoGII M.CviPII; M.EcoGII M.CviQIX; M.EcoGII M.CviQVIII; M.EcoGII M.CviQX; M.CvQII M.CviPI; M.CvQII M.CviPII; M.CvQII M.CviQIX; M.CvQII M.CviQVIII; and M.CvQII M.CviQX. In some embodiments, the methyltransferases are one of the following combinations (demarcated by semicolon): M.EcoKDam M.CviPI; M.Tsp509I M.CviPI; M.TaqI M.CviPI; M.CviQI M.CviPI; M.AluI M.EcoGII; M.AluI M.EcoKDam; M.HhaI M.EcoGII; M.HhaI M.EcoKDam; M.HpaII M.EcoGII; M.HpaII M.EcoKDam; M.HaeIII M.EcoGII; M.HaeIII M.EcoKDam; M.Sau3AI M.EcoGII; M.Sau3AI M.EcoKDam; M.AluI M.Tsp509I; M.HaeIII M.Tsp509I; M.HhaI M. Tsp509I; M.HpaII M.Tsp509I; M.MspI M.Tsp509I; M.Sau3AI M.Tsp509I; M.AluI M.HaeIII M. Tsp509I; M.AluI M.HhaI M.Tsp509I; M.AluI M.HpaII M.Tsp509I; M.AluI M.HhaI M. Tsp509I; M.AluI M.HpaII M.Tsp509I; M.AluI M.Sau3AI M.Tsp509I; M.HhaI M.Sau3A M.Tsp509I; M.AluI M.HhaI M.Sau3A M.Tsp509I; M.AluI M.CviQI; M.HaeIII M.CviQI; M.HhaI M.CviQI; M.HpaII M.CviQI; M.MspI M.CviQI; M.Sau3AI M.CviQI; M.AluI M.HaeIII M.CviQI; M.AluI M.HhaI M.CviQI; M.AluI M.HpaII M.CviQI; M.AluI M.HhaI M.CviQI; M.AluI M.HpaII M.CviQI; M.AluI M.Sau3AI M.CviQI; M.HhaI M.Sau3A M.CviQI; M.AluI M.HhaI M.Sau3A M.CviQI; M.AluI M.HhaI M.EcoGII; M.AluI M.HhaI M.EcoKDam; M.AluI M.HpaII M.EcoGII; M.AluI M.HpaII M.EcoKDam; M.AluI M.HaeIII M.EcoGII; M.AluI M.HaeIII M.EcoKDam; M.AluI M.Sau3AI M.EcoGII; M.AluI M.Sau3AI M.EcoKDam; M.AluI M.EcoGII M.EcoKDam; M.HhaI M.HpaII M.EcoGII; M.HhaI M.HpaII M.EcoKDam; M.HhaI M.HaeIII M.EcoGII; M.HhaI M.HaeIII M.EcoKDam; M.HhaI M.Sau3AI M.EcoGII; M.HhaI M.Sau3AI M.EcoKDam; M.HhaI M.EcoGII M.EcoKDam; M.HpaII M.HaeIII M.Sau3AI; M.HpaII M.HaeIII M.EcoGII; M.HpaII M.HaeIII M.EcoKDam; M.HpaII M.Sau3AI M.EcoGII; M.HpaII M.Sau3AI M.EcoKDam; M.HpaII M.EcoGII M.EcoKDam; M.HaeIII M.Sau3AI M.EcoGII; M.HaeIII M.Sau3AI M.EcoKDam; M.HaeIII M.EcoGII M.EcoKDam; M.Sau3AI M.EcoGII M.EcoKDam; M.AluI M.HhaI M.HpaII M.EcoGII; M.AluI M.HhaI M.HpaII M.EcoKDam; M.AluI M.HhaI M.HaeIII M.EcoGII; M.AluI M.HhaI M.HaeIII M.EcoKDam; M.AluI M.HhaI M.Sau3AI M.EcoGII; M.AluI M.HhaI M.Sau3AI M.EcoKDam; M.AluI M.HhaI M.EcoGII M.EcoKDam; M.AluI M.HpaII M.HaeIII M.EcoGII; M.AluI M.HpaII M.HaeIII M.EcoKDam; M.AluI M.HpaII M.Sau3AI M.EcoGII; M.AluI M.HpaII M.Sau3AI M.EcoKDam; M.AluI M.HpaII M.EcoGII M.EcoKDam; M.AluI M.HaeIII M.Sau3AI M.EcoGII; M.AluI M.HaeIII M.Sau3AI M.EcoKDam; M.AluI M.HaeIII M.EcoGII M.EcoKDam; M.AluI M.Sau3AI M.EcoGII M.EcoKDam; M.HhaI M.HpaII M.HaeIII M.EcoGII; M.HhaI M.HpaII M.HaeIII M.EcoKDam; M.HhaI M.HpaII M.Sau3AI M.EcoGII; M.HhaI M.HpaII M.Sau3AI M.EcoKDam; M.HhaI M.HpaII M.EcoGII M.EcoKDam; M.HhaI M.HaeIII M.Sau3AI M.EcoGII; M.HhaI M.HaeIII M.Sau3AI M.EcoKDam; M.HhaI M.HaeIII M.EcoGII M.EcoKDam; M.HhaI M.Sau3AI M.EcoGII M.EcoKDam; M.HpaII M.HaeIII M.Sau3AI M.EcoGII; M.HpaII M.HaeIII M.Sau3AI M.EcoKDam; M.HpaII M.HaeIII M.EcoGII M.EcoKDam; M.HpaII M.Sau3AI M.EcoGII M.EcoKDam; M.HaeIII M.Sau3AI M.EcoGII M.EcoKDam; M.AluI M.HhaI M.HpaII M.HaeIII M.EcoGII; M.AluI M.HhaI M.HpaII M.HaeIII M.EcoKDam; M.AluI M.HhaI M.HpaII M.Sau3AI M.EcoGII; M.AluI M.HhaI M.HpaII M.Sau3AI M.EcoKDam; M.AluI M.HhaI M.HpaII M.EcoGII M.EcoKDam; M.AluI M.HhaI M.HaeIII M.Sau3AI M.EcoGII; M.AluI M.HhaI M.HaeIII M.Sau3AI M.EcoKDam; M.AluI M.HhaI M.HaeIII M.EcoGII M.EcoKDam; M.AluI M.HhaI M.Sau3AI M.EcoGII M.EcoKDam; M.AluI M.HpaII M.HaeIII M.Sau3AI M.EcoGII; M.AluI M.HpaII M.HaeIII M.Sau3AI M.EcoKDam; M.AluI M.HpaII M.HaeIII M.EcoGII M.EcoKDam; M.AluI M.HpaII M.Sau3AI M.EcoGII M.EcoKDam; M.AluI M.HaeIII M.Sau3AI M.EcoGII M.EcoKDam; M.HhaI M.HpaII M.HaeIII M.Sau3AI M.EcoGII; M.HhaI M.HpaII M.HaeIII M.Sau3AI M.EcoKDam; M.HhaI M.HpaII M.HaeIII M.EcoGII M.EcoKDam; M.HhaI M.HpaII M.Sau3AI M.EcoGII M.EcoKDam; M.HhaI M.HaeIII M.Sau3AI M.EcoGII M.EcoKDam; M.HpaII M.HaeIII M.Sau3AI M.EcoGII M.EcoKDam; M.AluI M.HhaI M.HpaII M.HaeIII M.Sau3AI M.EcoGII; M.AluI M.HhaI M.HpaII M.HaeIII M.Sau3AI M.EcoKDam; M.AluI M.HhaI M.HpaII M.HaeIII M.EcoGII M.EcoKDam; M.AluI M.HhaI M.HpaII M.Sau3AI M.EcoGII M.EcoKDam; M.AluI M.HhaI M.HaeIII M.Sau3AI M.EcoGII M.EcoKDam; M.AluI M.HpaII M.HaeIII M.Sau3AI M.EcoGII M.EcoKDam; M.HhaI M.HpaII M.HaeIII M.Sau3AI M.EcoGII M.EcoKDam; and M.AluI M.HhaI M.HpaII M.HaeIII M.Sau3AI M.EcoGII M.EcoKDam.

In some embodiments, the at least one methyltransferase methylates at least a certain percentage of adenine, non-cytosine, and/or cytosine nucleotides contained within a sample. The percentage of nucleotides that any one methyltransferase methylates is calculated based on all the sequences present within a sample. The sequences present within a sample can be determined de novo by sequencing or estimated based on published sequences in public databases. In the case of samples derived from organisms with published genomes, the percentage of the nucleotides is calculated in the following manner.

When a sample is of human origin, the percentage is calculated using the published human genome, particularly NCBI build 38.2 (GRCh38.p2). All the chromosome localized contigs of the primary assembly are used which include scaffolds (NT ids) and patches (NW ids). Scaffolds which were localized only to a particular chromosome are also counted. Alternate loci are skipped (sequences with ALT_REF_LOCI in the description line). Ambiguous nucleotides such as Ns were not added to the base total (length). The total number of non-N bases counted for the human genome (GRCh38.p2) was 2,956,425,695; of these, 2,956,425,596 were A or C or G or T.

Methyltransferase recognition sites varied in size from 1 to 6 bases long. All were counted as single entities for the purposes of percentage methylation calculations. They were not normalized per length of site as the enzymes introduce methylation at a single nucleotide within the recognition sequence. Site matching in the human genome was done with the exact enzyme recognition sequence, and no ambiguous bases were matched. For example, for the dcm site with a CCWGG consensus sequence, counts are performed for CCAGG and CCTGG separately and summed to get the total for CCWGG. Counts were for the number of occurrences of each site over the length of a genomic contig and then totaled over the entire genome. Counts are reported in Table 1 below as number/kb which is the total number of sites counted in the genome divided by the total number of bases counted in the genome multiplied by 1000 (the “Mean num sites/1 kb” column in Table 1) or as a percent which is the total number of sites counted in the genome divided by the total number of bases counted in the genome multiplied by 100 (the “Mean % sites (num/100 bases)” column in Table 1). For any enzyme or enzyme combination not listed in Table 1, the percentage methylation calculations are carried out in accordance with the method described herein that was used to generate the calculations shown in Table 1. This same approach can be applied to other non-human organisms with published genomes.

For instance, the frequency of methylation of EcoGII is estimated at ≥50% of all A nucleotides in the genome. The percentage of adenine nucleotides is 29.51 in the human genome. Accordingly, the minimum percent of methylated adenines that would be expected after treatment with M.EcoGII is ≥14.8 (or ≥148/kb). M.EcoGII can also be used to introduce methylation at less than 14.8% of nucleotides by limiting the amount of enzyme or co-factors that are added to the methylation reaction. Adenine methyltransferases with specific recognition sequences may be used in conjunction with the M.EcoGII methyltransferase to enable the verification of the methylation reaction using methylation sensitive restriction endonucleases. For the calculation of methylation frequency when EcoGII is used in combination with other enzymes that methylate adenine residues (e.g., dam, M.TaqI, AACCA, ACATC, M.EcoRI, GAWTC, and SATC enzymes), only the EcoGII frequency is included in the final calculation because of the overlap in the recognition sequences. In other words, for the purpose of calculating the frequency of methylation, if M.EcoGII is present in a combination with any other adenine methyltransferases, the frequency of methylated adenines per kb is that of M.EcoGII, i.e., ≥148/kb, because M.EcoGII could potentially methylate all the sites methylated by other adenine methyltransferases. However, if EcoGII is used in combination with one or more cytosine methyltransferases, then the sum of the methylation frequencies of the one or more cytosine methyltransferase would be added to the expected methylation frequency of the M.EcoGII methyltransferase, as expressed in Table 1 as “Mean % sites (num/100 bases).” For all other enzyme combinations, the calculation of methylation frequency is the sum of the “Mean % sites (num/100 bases)” listed in Table 1 for each enzyme within the combination. For instance, the methylation frequency for a combination of BamHI and AluI would yield a total of 0.45%, i.e., 0.01%+0.44%.

TABLE 1

| | | | Mean | Mean % |
|---|---|---|---|---|
| | | | num | sites |
| Enzyme name | Site | Base | sites/ | (num/100 |
| or combination | sequence | methylated | 1 kb | bases) |
| M.AluI | AGCT | C | 4.44 | 0.44 |
| M.BamHI | GGATCC | C | 0.12 | 0.01 |
| M.EcoKdcm | CCWGG | C | 3.36 | 0.34 |
| M.HaeIII | GGCC | C | 2.92 | 0.29 |
| M.HhaI | GCGC | C | 0.58 | 0.06 |
| M.HpaII | CCGG | C | 0.79 | 0.08 |
| M.MspI | CCGG | C | 0.79 | 0.08 |
| M.Sau3AI | GATC | C | 2.45 | 0.25 |
| M.NspI | RCATGY | C | 0.55 | 0.06 |
| phi3T | GGCC/GCNGC | C | 4.97 | 0.5 |
| spr | GGCC/CCGG/ | C | 7.07 | 0.71 |
| | CCWGG | | | |
| M.CviPI | GC | C | 42.43 | 4.24 |
| M.CviQI | GTAC | A | 1.74 | 0.17 |
| AACCA | AACCA | A | 1.14 | 0.11 |
| ACATC | ACATC | A | 0.87 | 0.09 |
| GAWTC | GAWTC | A | 1.71 | 0.17 |
| M.EcoKdam | GATC | A | 2.45 | 0.25 |
| M.EcoGII | A | A | >147.5 | >14.8 |
| M.EcoRI | GAATTC | A | 0.29 | 0.03 |
| GAWTC | GAWTC | A | 1.71 | 0.17 |
| SATC | SATC | A | 5.94 | 0.6 |
| M. TaqI | TCGA | A | 0.55 | 0.05 |
| M.Tsp509I | AATT | A | 7.4 | 0.74 |
| M1.Bst19I | GCATC | A | 0.62 | 0.06 |
| AluI + HaeIII | | C | 7.36 | 0.73 |
| AluI + dam + | | C/A | 7.47 | 0.75 |
| HhaI | | | | |
| AluI + dcm | | C | 7.8 | 0.78 |
| SATC + dcm | | C/A | 9.3 | 0.94 |
| AluI + HaeIII + | | C/A | 9.81 | 0.98 |
| dam | | | | |
| HaeIII + SATC + | | C/A | 10 | 1 |
| AACCA | | | | |
| AluI + SATC | | C/A | 10.38 | 1.04 |
| AluI + HaeIII + | | C | 10.72 | 1.07 |
| dcm | | | | |
| AluI + phi3T + | | C/A | 10.75 | 1.07 |
| TaqI + HpaII | | | | |
| SATC + phi3T | | C/A | 10.91 | 1.1 |
| AluI + dam + | | C/A | 10.83 | 1.09 |
| HhaI + dcm | | | | |
| AluI + SATC + | | C/A | 10.93 | 1.09 |
| TaqI | | | | |
| AluI + GAWTC + | | C/A | 11.12 | 1.11 |
| phi3T | | | | |
| AluI + SATC + | | C/A | 11.52 | 1.15 |
| AACCA | | | | |
| AluI + HaeIII + | | C/A | 13.3 | 1.33 |
| SATC | | | | |
| AluI + SATC + | | C/A | 13.74 | 1.38 |
| dcm | | | | |
| EcoGII + BamHI | | C/A | ≥147.62 | ≥14.81 |
| EcoGII + HaeIII | | C/A | ≥150.42 | ≥15.09 |
| EcoGII + dcm | | C/A | ≥150.86 | ≥15.14 |
| EcoGII + AluI | | C/A | ≥151.94 | ≥15.24 |
| EcoGII + phi3T | | C/A | ≥152.47 | ≥15.3 |
| EcoGII + AluI + | | C/A | ≥152.52 | ≥15.3 |
| HhaI | | | | |
| EcoGII + AluI + | | C/A | ≥154.86 | ≥15.53 |
| HaeIII | | | | |

As used herein, when the phrase “at least one methyltransferase” is used without further indication whether the at least one methyltransferase methylates adenine or cytosine nucleotides, the phrase refers to at least one methyltransferase that methylates adenine nucleotides, at least one methyltransferase that methylates cytosine nucleotides, or a combination thereof.

Nucleotide symbols used herein correspond with the listing of nucleotides found in Annex I, Section 1 of “Standard ST.26” published by WIPO (approved Nov. 5, 2021). Particularly for DNA nucleotides, A is adenine, C is cytosine, G is guanine, t is thymine, M is A or C, R is A or G, W is A or T, S is C or G, Y is C or T, K is G or T, V is A or C or G (i.e., not T), H is A or C or T (i.e., not G), D is A or G or T (i.e., not C), B is C or G or T (i.e., not A), and N is A or C or G or T.

In some embodiments wherein the at least one methyltransferase includes at least one methyltransferase that methylates adenine nucleotides, the at least one methyltransferase methylates at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10%, at least 20%, at least 30%, at least 40%, or at least 50% of nucleotides in the sample. In an embodiment, the at least one methyltransferase methylates at least 0.3% of nucleotides in the sample. In an embodiment, the at least one methyltransferase methylates at least 2% of nucleotides in the sample. In an embodiment, the at least one methyltransferase methylates at least 5% of nucleotides in the sample. In an embodiment, the at least one methyltransferase methylates at least 10% of nucleotides in the sample.

In some embodiments wherein the at least one methyltransferase includes at least one methyltransferase that methylates adenine nucleotides, the at least one methyltransferase methylates any suitable percentage range of nucleotides in the sample, including, for instance, 0.3-1%, 0.3-2%, 0.3-5%, 0.3-10%, 0.3-20%, 0.3-30%, 0.3-40%, 0.3-50%, 0.4-1%, 0.4-2%, 0.4-5%, 0.4-10%, 0.4-20%, 0.4-30%, 0.4-40%, 0.4-50%, 0.5-1%, 0.5-2%, 0.5-5%, 0.5-10%, 0.5-20%, 0.5-30%, 0.5-40%, 0.5-50%, 0.6-1%, 0.6-2%, 0.3-5%, 0.6-10%, 0.6-20%, 0.6-30%, 0.6-40%, 0.6-50%, 0.7-1%, 0.7-2%, 0.3-5%, 0.7-10%, 0.7-20%, 0.7-30%, 0.7-40%, 0.7-50%, 0.8-1%, 0.8-2%, 0.8-5%, 0.8-10%, 0.8-20%, 0.8-30%, 0.8-40%, 0.8-50%, 0.9-1%, 0.9-2%, 0.9-5%, 0.9-10%, 0.9-20%, 0.9-30%, 0.9-40%, 0.9-50%, 0.9-1%, 0.9-2%, 0.9-5%, 0.9-10%, 0.9-20%, 0.9-30%, 0.9-40%, 0.9-50%, 1-2%, 1-5%, 1-10%, 1-20%, 1-30%, 1-40%, and 1-50%, of the nucleotides in the sample. In certain embodiments, the percentage range is between any two percentages disclosed in the preceding paragraph, e.g., 5-6%.

In some embodiments wherein the at least one methyltransferase includes at least one methyltransferase that methylates non-cytosine nucleotides, the at least one methyltransferase methylates at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 20%, at least 30%, at least 40%, or at least 50% of nucleotides in the sample. In an embodiment, the at least one methyltransferase methylates at least 2% of nucleotides in the sample. In an embodiment, the at least one methyltransferase methylates at least 5% of nucleotides in the sample. In an embodiment, the at least one methyltransferase methylates at least 10% of nucleotides in the sample. In an embodiment, the at least one methyltransferase methylates at least 0.3% of nucleotides in the sample.

In some embodiments wherein the at least one methyltransferase includes at least one methyltransferase that methylates non-cytosine nucleotides, the at least one methyltransferase methylates any suitable percentage range of nucleotides in the sample, including, for instance, 0.3-1%, 0.3-2%, 0.3-5%, 0.3-10%, 0.3-20%, 0.3-30%, 0.3-40%, 0.3-50%, 0.4-1%, 0.4-2%, 0.4-5%, 0.4-10%, 0.4-20%, 0.4-30%, 0.4-40%, 0.4-50%, 0.5-1%, 0.5-2%, 0.5-5%, 0.5-10%, 0.5-20%, 0.5-30%, 0.5-40%, 0.5-50%, 0.6-1%, 0.6-2%, 0.3-5%, 0.6-10%, 0.6-20%, 0.6-30%, 0.6-40%, 0.6-50%, 0.7-1%, 0.7-2%, 0.7-5%, 0.7-10%, 0.7-20%, 0.7-30%, 0.7-40%, 0.7-50%, 0.8-1%, 0.8-2%, 0.8-5%, 0.8-10%, 0.8-20%, 0.8-30%, 0.8-40%, 0.8-50%, 0.9-1%, 0.9-2%, 0.9-5%, 0.9-10%, 0.9-20%, 0.9-30%, 0.9-40%, 0.9-50%, 0.9-1%, 0.9-2%, 0.9-5%, 0.9-10%, 0.9-20%, 0.9-30%, 0.9-40%, 0.9-50%, 1-2%, 1-5%, 1-10%, 1-20%, 1-30%, 1-40%, and 1-50%, of the nucleotides in the sample. In certain embodiments, the percentage range is between any two percentages disclosed in the preceding paragraph, e.g., 5-6%.

In some embodiments, the at least one methyltransferase methylates at least 0.1%, at least 0.2%, at least 0.3%, at least 0.4%, at least 0.5%, at least 0.6%, at least 0.7%, at least 0.71%, at least 0.72%, at least 0.73%, at least 0.74%, at least 0.75%, at least 0.76%, at least 0.77%, at least 0.78%, at least 0.79%, at least 0.8%, at least 0.9%, at least 1%, at least 2%, at least 3%, at least 4%, at least 5%, at least 6%, at least 7%, at least 8%, at least 9%, or at least 10%, at least 20%, at least 30%, at least 40%, or at least 50% of the nucleotides in the sample. In certain embodiments, the at least one methyltransferase methylates a certain percentage range of the nucleotides in the sample, wherein the range is between any two percentages disclosed in the preceding paragraph, e.g., 5-6%. In an embodiment, the at least one methyltransferase methylates at least 0.7% of nucleotides in the sample. In an embodiment, the at least one methyltransferase methylates at least 0.71% of nucleotides in the sample. In an embodiment, the at least one methyltransferase methylates at least 0.72% of nucleotides in the sample. In an embodiment, the at least one methyltransferase methylates at least 0.73% of nucleotides in the sample. In an embodiment, the at least one methyltransferase methylates at least 0.74% of nucleotides in the sample. In an embodiment, the at least one methyltransferase methylates at least 0.75% of nucleotides in the sample. In an embodiment, the at least one methyltransferase methylates at least 0.76% of nucleotides in the sample. In an embodiment, the at least one methyltransferase methylates at least 0.77% of nucleotides in the sample. In an embodiment, the at least one methyltransferase methylates at least 0.78% of nucleotides in the sample. In an embodiment, the at least one methyltransferase methylates at least 0.79% of nucleotides in the sample. In an embodiment, the at least one methyltransferase methylates at least 0.8% of nucleotides in the sample. In an embodiment, the at least one methyltransferase methylates at least 2% of nucleotides in the sample. In an embodiment, the at least one methyltransferase methylates at least 5% of nucleotides in the sample. In an embodiment, the at least one methyltransferase methylates at least 10% of nucleotides in the sample.

In some embodiments wherein the at least one methyltransferase includes at least one methyltransferase that methylates non-cytosine nucleotides, the at least one methyltransferase methylates any suitable percentage range of nucleotides in the sample, including, for instance, 0.3-1%, 0.3-2%, 0.3-5%, 0.3-10%, 0.3-20%, 0.3-30%, 0.3-40%, 0.3-50%, 0.4-1%, 0.4-2%, 0.4-5%, 0.4-10%, 0.4-20%, 0.4-30%, 0.4-40%, 0.4-50%, 0.5-1%, 0.5-2%, 0.5-5%, 0.5-10%, 0.5-20%, 0.5-30%, 0.5-40%, 0.5-50%, 0.6-1%, 0.6-2%, 0.3-5%, 0.6-10%, 0.6-20%, 0.6-30%, 0.6-40%, 0.6-50%, 0.7-1%, 0.7-2%, 0.7-5%, 0.7-10%, 0.7-20%, 0.7-30%, 0.7-40%, 0.7-50%, 0.71-1%, 0.71-2%, 0.71-5%, 0.71-10%, 0.71-20%, 0.71-30%, 0.71-40%, 0.71-50%, 0.72-1%, 0.72-2%, 0.72-5%, 0.72-10%, 0.72-20%, 0.72-30%, 0.72-40%, 0.72-50%, 0.73-1%, 0.73-2%, 0.73-5%, 0.73-10%, 0.73-20%, 0.73-30%, 0.73-40%, 0.73-50%, 0.74-1%, 0.74-2%, 0.74-5%, 0.74-10%, 0.74-20%, 0.74-30%, 0.74-40%, 0.74-50%, 0.75-1%, 0.75-2%, 0.75-5%, 0.75-10%, 0.75-20%, 0.75-30%, 0.75-40%, 0.75-50%, 0.76-1%, 0.76-2%, 0.76-5%, 0.76-10%, 0.76-20%, 0.76-30%, 0.76-40%, 0.76-50%, 0.77-1%, 0.77-2%, 0.77-5%, 0.77-10%, 0.77-20%, 0.77-30%, 0.77-40%, 0.77-50%, 0.78-1%, 0.78-2%, 0.78-5%, 0.78-10%, 0.78-20%, 0.78-30%, 0.78-40%, 0.78-50%, 0.79-1%, 0.79-2%, 0.79-5%, 0.79-10%, 0.79-20%, 0.79-30%, 0.79-40%, 0.79-50%, 0.8-1%, 0.8-2%, 0.8-5%, 0.8-10%, 0.8-20%, 0.8-30%, 0.8-40%, 0.8-50%, 0.9-1%, 0.9-2%, 0.9-5%, 0.9-10%, 0.9-20%, 0.9-30%, 0.9-40%, 0.9-50%, 0.9-1%, 0.9-2%, 0.9-5%, 0.9-10%, 0.9-20%, 0.9-30%, 0.9-40%, 0.9-50%, 1-2%, 1-5%, 1-10%, 1-20%, 1-30%, 1-40%, or 1-50%, of the nucleotides in the sample. In certain embodiments, the percentage range is between any two percentage values disclosed in the preceding paragraph, e.g., 5-6%.

In certain embodiments, the methods of the invention include an assaying step, in which the sample, purified nucleic acid, and/or tagged nucleic acid is assayed for the methylation status of one or more cytosine nucleotides in the at least one target sequence. The assay for methylation status can employ any suitable assay known in the art. In some embodiments, the assay step includes digesting the sample or purified DNA or tagged DNA with restriction endonucleases, preferably sequence-specific restriction endonucleases. In other embodiments, the assay step includes using chemicals, enzymes or a combination of enzymes and chemicals to differentiate unmodified from modified cytosines, such as 5-methyl or 5-hydroxymethyl-cytosines (Frommer et al., “A genomic sequencing protocol that yields a positive display of 5-methylcytosine residues in individual DNA strands,” Proc. Natl. Acad. Sci. U.S.A., 89(5): 1827-1831 (1992); Shiraishi and Hayatsu “High-speed conversion of cytosine to uracil in bisulfite genomic sequencing analysis of DNA methylation.” DNA res. 11(6): 409-15 (2004); Hayatsu et al. “Progress in the bisulfite modification of nucleic acids,” Nucleic Acids Symp Ser (Oxf) 53: 217-218(2009); Ito et al., “Family-Wide Comparative Analysis of Cytidine and Methylcytidine Deamination by Eleven Human APOBEC Proteins,” J. Mol. Biol., 429(12): 1787-1799 (2017); Schutsky et al., “Nondestructive, base-resolution sequencing of 5-hydroxymethylcytosine using a DNA deaminase,” Nat. Biotechnol., 36: 1083-1090 (2018); Yu et al., “Tet-Assisted Bisulfite Sequencing (TAB-seq),” Methods Mol, Biol., 1708: 645-663 (2018); Liu et al., “Bisulfite-free direct detection of 5-methylcytosine and 5-hydroxymethylcytosine at base resolution,” Nat. Biotechnol., 37(4): 424-429 (2019)).

In other embodiments, the assay step includes single nucleotide primer extension, termination-coupled linear amplification, combined bisulfite restriction analysis (COBRA), methylation-specific PCR, methylation-specific quantitative PCR, pyrosequencing, droplet-digital PCR, mass spectrometry methylation-sensitive high resolution melting analysis (MS-HRM), headloop suppression PCR, ligation-mediated amplification, bisulfite patch PCR, methylation-specific quantum fluorescent resonance energy transfer (MS-qFRET), microarray analysis, bead hybridization (e.g. X-MAP™ technology from Luminex corporation), mass spectrometry, or any combination thereof (Kurdyukov et Bullock, “DNA methylation analysis: choosing the right method,” Biology 5(1): 3 (2016); Fraga and Esteller “DNA methylation: a profile of methods and applications,” Biotechniques 33(3): 632 (2002); Cottrell and Laird “Sensitive detection of DNA methylation,” Ann N Y Acad Sci. 983: 120-30 (2003); Wong “Qualitative and quantitative polymerase chain reaction-based methods for DNA methylation analysis,” Methods Mol. Biol. 336: 33-34 (2006); Xiong et al., “COBRA: a sensitive and quantitative DNA methylation assay,” Nucleic Acids Res., 25(12): 2532-2534 (1997); Gonzalgo et al., “Rapid quantitation of methylation differences at specific sites using methylation-sensitive single nucleotide primer extension (Ms-SNuPE),” Nucleic Acids Res., 25(12): 2529-2531 (1997); Rand et al. “Headloop suppression PCR and its application to selective amplification of methylated DNA sequences,” Nuclei Acids Res. 33(14):e127 (2005); Wojdacz et al., “Methylation-sensitive high resolution melting (MS-HRM): a new approach for sensitive and high-throughput assessment of methylation,” Nucleic Acids Res., 35(6): e41 (2007); Varley et al., “Bisulfite Patch PCR enables multiplexed sequencing of promoter methylation across cancer samples,” Genome Res., 20(9): 1279-1287 (2010); Bailey et al., “MS-qFRET: a quantum dot-based method for analysis of DNA methylation,” Genome Res., 19(8): 1455-1461 (2009); Yu et al. “MethyLight droplet digital PCR for detection and absolute quantification of infrequently methylated alleles,” Epigenetics 10(9): 803-9 (2015); Ehrich et al. “Quantitative high-throughput analysis of DNA methylation patterns by base-specific cleavage and mass spectrometry,” Proc. Natl. Acad. Sci. U.S.A. 102(44): 15785-15790 (2005); Karimi et al. “Using LUMA: a luminometric-based assay for global DNA-methylation,’ Epigenetics 1(1): 45-8 (2006); Yan et al. “Dissecting complex epigenetic alterations in breast cancer using CpG island microarrays,” Cancer Res 61:8375-8380 (2001); Bibikova et al., “High-throughput DNA methylation profiling using universal bead arrays,” Genome Res., 16(3): 383-393 (2006); Bibikova et al., “High density DNA methylation array with single CpG site resolution,” Genomics, 98(4): 288-295 (2011)).

In other embodiments, the assay steps include using targeted methylation sequencing, next generation sequencing, array-capture bisulfite sequencing, bisulfite padlock probes, or any combination thereof. Methylation sequencing may include whole genome sequencing of bisulfite or enzymatically modified DNA or sequencing of targeted genomic fragments (Lee et al. “Analyzing the cancer methylome through targeted bisulfite sequencing,” Cancer Lett. 340(2): 171-8 (2013); Bentley et al., “Accurate whole human genome sequencing using reversible terminator chemistry,” Nature 456(7218): 53-59 (2008); Cokus et al., “Shotgun bisulphite sequencing of the Arabidopsis genome reveals DNA methylation patterning,” Nature, 452(7184): 215-219 (2008); Lister et al., “Human DNA methylomes at base resolution show widespread epigenomic differences,” Nature, 462(7271): 315-322 (2009); Harris et al. “Comparison of sequencing-based methods to profile DNA methylation and identification of monoallelic epigenetic modifications,” Nat. Biotechnol. 28(10: 1097-105 (2010); Bock et al. “Quantitative comparison of genome-wide DNA methylation mapping technologies,” Nat. biotechnol. 28(10): 1106-14 (2010); Booth et al. “Oxidative bisulfite sequencing of 5-methylcytosine and 5-hydroxymethylcytosine”, Nat. Protocol 8(10): 1841-51 (2013); Vaisvila et al. “Enzymatic methyl sequencing detects DNA methylation at single-base resolution from picograms of DNA,” Genome Res. 1(7): 1280-1289 (2021); Liu et al., “Accurate targeted long-read DNA methylation and hydroxymethylation sequencing with TAPS,” Genome Biol., 21(1): 54 (2020); Liu et al., “Subtraction-free and bisulfite-free specific sequencing of 5-methylcytosine and its oxidized derivatives at base resolution,” Nat. Commun., 12(1): 618 (2021); Diep et al. “Library-free methylation sequencing with bisulfite padlock probes,” Nat. Methods 9(3): 270-2 (2012); Komori et al. “application of microdroplet PCR for large scale targeted bisulfite sequencing,” Genome res. 21: 1738-45 (2011); Hodges et al. “High definition profiling of mammalian DNA methylation by array-capture and single molecule bisulfite sequencing,” Genome Res. 19: 1593-1605 (2009)).

In other embodiments, the assay step includes third generation direct sequencing of DNA and the detection of modified nucleotides (White and Hesselberth. “Modification mapping by nanoprore sequencing,” Front. Genet., 13: 1037134 (2022), Wang et al. “Nanopore sequencing technology, bioinformatics and applications,” Nat Biotechnol. 39(11): 1348-1365 (2021), FOOX et al. “The SEQC2 epigenomics quality control (EpiQC) study,” Genome Biol. 22: 232 (2021), Tse et al. “Genome wide detection of cytosine methylation by single molecule real-time sequencing,” PNAS 118(5): e2019768118)). When analyzing multiple markers, the methods of the invention can include, for instance, techniques such as multiplex PCR, nested PCR and the use of modified or degenerate primers. For example, primers can be degenerate at the position corresponding to one or more cytosine that are expected to be methylated within the target sequence due to in vivo or in vitro methylation. Such primers can be used to maximize the amplification of all templates present within a sample or to maximize the amplification of all methylated templates within the sample.

The methods of the invention can be used to evaluate the methylation status of a CpG island having a methylation status that is associated with a disease state such as cancer. Non-invasive diagnostic, predictive, or prognostic tests as well as tests to monitor response to therapy may require evaluating multiple methylated markers and potentially multiple assays for individual markers (Bettegowda et al., “Detection of circulating tumor DNA in early- and late-stage human malignancies,” Sci. Transl. Med., 6(224): 224ra24 (2014); Diehl et al., “Circulating mutant DNA to assess tumor dynamics,” Nat. Med., 14(9): 985-990 (2008); Ossandon et al., “Circulating Tumor DNA Assays in Clinical Cancer Research,” J. Natl. Cancer Inst., 110(9): 929-934 (2018); Tie et al., “Circulating tumor DNA analysis detects minimal residual disease and predicts recurrence in patients with stage II colon cancer,” Sci. Transl. Med., 8(346): 346ra92 (2016)). Therefore, clinical tests based on nucleic acid methylation may require the analysis of multiple CpG islands, multiple CpG dinucleotides across the length of an island or multiple cytosines in one or more RNA molecules. Inasmuch as they avoid depleting the sample, the methods of the invention can be used to evaluate the methylation status of multiple target sequences in samples that include relatively small amounts of DNA or RNA such as nucleic acids recovered from bodily fluids.

The methods of the invention can be used to evaluate the methylation status of 1 or more, 5 or more, 10 or more, 100 or more, 1000 or more, or 10000 or more target sequences in a sample. In some embodiments, the sample includes a relatively small amount of genomic DNA, such as 15 ng or less. Thus, for example, the methods of the invention can be used to evaluate circulating DNA or RNA from plasma or urine for the methylation status of multiple (e.g., 5 or more, 10 or more, 100 or more, 1000 or more, 10000 or more) cytosines associated with a disease, such as cancer. The disease can be any cancer, including leukemia (e.g., lymphoblastic, myeloid, hairy cell), adrenocortical carcinoma, anal cancer, appendix cancer, astrocytoma, basal cell carcinoma, extrahepatic bile duct cancer, bladder cancer, bone cancer, brain cancer, gliomas, breast cancer, bronchial adenomas, carcinoid tumors, cervical cancer, myeloproliferative disorders, colon cancer, endometrial cancer, esophageal cancer, eye cancer, gallbladder cancer, stomach cancer, gastrointestinal tumors, head and neck cancer, liver cancer, lymphomas (e.g., Hodgkin's, Non-Hodgkin's, Burkitt's, T-cell, central nervous system, and AIDS-related), sarcomas, kidney cancer, laryngeal cancer, lip and oral cavity cancer, liver cancer, lung cancer, macroglobulinemia, melanoma, Merkel cell carcinoma, mesothelioma, myeloproliferative disorders, nasal and paranasal sinus cancer, nasopharyngeal cancer, neuroblastoma, ovarian cancer, pancreatic cancer, parathyroid cancer, penile cancer, pharyngeal cancer, pheochromocytoma, pituitary tumor, pancreatic cancer, prostate cancer, rectal cancer, kidney cancer, salivary gland cancer, skin cancer (non-melanoma), testicular cancer, throat cancer, thyroid cancer, trophoblastic tumor ureter and renal pelvis cancer, urethral cancer, uterine cancer, vaginal cancer, and vulvar cancer. In certain embodiments, the disease is lung cancer, prostate cancer, ovarian cancer, colon cancer, liver cancer, pancreatic cancer, thyroid cancer, skin cancer, head and neck cancer, brain cancer, or hematological cancer. In certain embodiments, the disease is prostate cancer. The methods of the invention can also be used to evaluate other diseases including infectious, immunological, and neurological diseases.

In certain embodiments, the methods of the invention can be used to evaluate the methylation status of one or more CpG islands associated with one or more of the following genes: ATP binding cassette subfamily B member 1 (ABCB1, ID 5243), ATP binding cassette subfamily C member 1 (ABCC1, ID 4363), ADAM metallopeptidase domain 23 (ADAM23, ID: 8745), adenylate cyclase 4 (ADCY4, ID 196883), adenylate cyclase 8 (ADCY8, ID 114), aldehyde oxidase 1 (AOX1, ID: 316), ankyrin repeat domain 13B (ANKRD13B, ID: 124930), APC regulator of WNT signaling pathway (APC, ID: 324), BCL2/adenovirus E1B 19 kDa interacting protein 3 (BNIP3, ID: 664), bone morphogenetic protein 3 (BMP3, ID: 651), BRCA1 DNA repair associated (BRCA1, ID: 672), BRCA2 DNA repair associated (BRCA2, ID: 675), cAMP-dependent protein kinase inhibitor alpha (PKIA, ID: 5569), checkpoint kinase 2 (CHK2, ID11200), C-X-C motif chemokine receptor 4 (CXCR4, ID 7852), C-X-C motif chemokine ligand 1 (CXCL1, ID 2919), C-X-C motif chemokine ligand 2 (CXCL2, ID 2920), C-X-C motif chemokine ligand 12 (CXCL12, ID 6387), C-X-C motif ligand chemokine 14 (CXCL14, ID: 9547), C-X-C motif chemokine ligand 16 (CXCL12, ID 58191), cyclin dependent kinase inhibitor 2A (CDKN2A, ID: 1029), carbohydrate sulfotransferase 2 (CHST2, ID: 9435), cyclin and CBS domain divalent metal cation transport mediator 1 (CNNM1, ID: 26507), cytochrome b-245 alpha chain (CYBA, ID: 1535), dedicator of cytokinesis 2 (DOCK2, ID: 1794), deltex E3 ubiquitin ligase 1 (DTX1, ID: 1840), dickkopf homolog 3 (DKK3, ID: 27122), embryonal Fyn-associated substrate (EFS, ID: 10278), epoxide hydrolase 1 (EPHX1, ID: 2052), epoxide hydrolase 3 (EPHX3, ID: 79852), fermitin family member 3 (FERMT3, ID: 83706), fibroblast growth factor 3 (FGF4, ID: 2248), fibroblast growth factor 4 (FGF4, ID: 2249), fibroblast growth factor 20 (FGF20, ID: 26281), fibroblast growth factor receptor 1 (FGFR1, ID: 2260), Fli-1 proto-oncogene, ETS transcription factor (FLII, ID: 2314), fms related receptor tyrosine kinase 4 (FLT4), forkhead box C1 (FOXC1, ID 2296), forkhead box E1 (FOXE1, ID: 2304), frizzled related protein (FRZB, ID: 2487), GATA binding protein 4 (GATA4, ID2626), GATA binding protein 5 (GATA5, ID140628), GDNF family receptor alpha 1 (GFRA1, ID: 2674), GDNF family receptor alpha2 (GFRA2, ID: 2675), G-protein coupled receptor 62 (GPR62, ID: 118442), glutamate ionotropic receptor NMDA type subunit 2D (GRIN2D, ID: 2906), glutathione peroxidase 1 (GPX1, ID 2876), glutathione peroxidase 3 (GPX3, ID: 2878), glutathione peroxidase 4 (GPX4, ID 2879), glutathione peroxidase 7 (GPX7, ID: 2882), glutathione S-transferase mu 3 (GSTM3, ID:2947), glutathione S-transferase 01 (GSTO1, ID: 9446), glutathione S-transferase O2 (GSTO2, ID: 119391), glutathione S-transferase P1 (GSTP1, ID: 2950), G protein-coupled receptor 62 (GPR62, ID: 118442), G protein-coupled receptor associated sorting protein 2 (GPRASP2, ID 114928), HOXA1 (ID: 3198), HOXA2 (ID: 3199), HOXA3 (ID: 3200), HOXA4 (ID: 3201), HOXA5 (ID: 3202), HOXA6 (ID: 3203), HOXA7 (ID: 3204), HOXA9 (ID: 3205), HOXA10 (ID: 3206), HOXA10-HOXA9 (ID: 100534589), HOXA11 (ID: 3207), HOXA13 (ID: 3209), HOXA-AS2 (ID: 285943), HOXA-AS3 (ID: 100133311), HOXA10-AS (ID: 100874323), HOXA11-AS (ID: 221883), MIR196B (ID: 442920), HOTAIRMI (ID: 100506311), HOTTIP (ID: 100316868), HOXB1 (ID: 3211), HOXB2 (ID: 3212), HOXB3 (ID: 3213), HOXB4 (ID: 3214), HOXB5 (ID: 3215), HOXB6 (ID: 3216), HOXB7 (ID: 3217), HOXB8 (ID: 3218), HOXB9 (ID: 3219), HOXB13 (ID: 10481), HOXB-AS1 (ID: 100874362), HOXB-AS2 (ID: 100874350), HOXB-AS3 (ID: 404266), HOXC4 (ID: 3221), HOXC5 (ID: 3222), HOXC6 (ID: 3223), HOXC8 (ID: 3224), HOXC9 (ID: 3225), HOXC10 (ID: 3226), HOXC11 (ID: 3227), HOXC12 (ID: 3228), HOXC13 (ID: 3229), HOXC-AS1 (ID: 100874363), HOXC-AS3 (ID: 100874365), HOXC13-AS (ID: 100874366), HOXD1 (ID: 3231), HOXD3 (ID: 3232), HOXD4 (ID: 3233), HOXD8 (ID: 3234), HOXD9 (ID: 3235), HOXD10 (ID: 3236), HOXD11 (ID: 3237), HOXD12 (ID: 3238), HOXD13 (ID: 3239), HAGLR (ID: 401022), HOXD-AS2 (ID: 100506783), hypoxia inducible factor 1 subunit alpha (HIF1A, ID: 3091), hypoxia inducible factor 3 subunit alpha (HIF3A, ID: 64344), junctional adhesion molecule 3 (JAM3, ID: 83700), junctophilin 3 (JPH3, ID: 57338), Kallikrein related peptidase (KLK10, ID: 5655), kinesin family member C2 (KIFC2, ID: 90990), leucine rich repeat containing 4 (LRRC4, ID: 64101), Meis homeobox 2 (MEIS2, ID: 4212), O-6-methylguanine-DNA methyltransferase (MGMT, ID: 4255), methyltransferase family member 1 (HEMK1, ID: 51409), monooxygenase DBH like 1 (MOXD1, ID: 26002), mutL homolog 1 (MLH1, ID: 4292), mutL homolog 3 (MLH3, ID: 27030), mutS homolog 2 (MSH2, ID: 4436), mutS homolog 6 (MSH6, ID: 2956), neurogenin 3 transcription factor (NEUROG3, ID: 50674), NDRG family member 4 (NDRG4, ID: 65009), nodal growth differentiation factor (NODAL, ID: 4838), oncostatin M receptor (OSMR, ID: 9180), 5-oxoprolinase, ATP-hydrolysing (OPLAH, ID: 26873), paired box 6 (PAX6, ID: 5080), paired like homeodomain 1 (PITX1, ID: 5307), paired like homeodomain 2 (PITX2, ID: 5308), paired related homeobox 1 (PRRX1, ID: 5396), partner and localizer of BRCA2 (PALB2, ID: 79728), phosphatase and actin regulator 3 (PHACTR3, ID: 116154), Phosphatase domain containing paladin 1 (PALD, ID: 27143), platelet derived growth factor D (PDGFD, ID: 80310), PR/SET domain 2 (PRDM2, ID: 7799), PR/SET domain 5 (PRDMS, ID: 11107), PR/SET domain 14 (PRDM14, ID: 63978), PR/SET domain 16 (PRDM16, ID: 63976), protein phosphatase 2 regulatory subunit Bbeta (PPP2R2B, ID: 5521), protein phosphatase 2 regulatory subunit Bgamma (PPP2R5C, ID: 5527), QKI, KH domain containing RNA binding (QKI, ID: 9444), Ras association domain family member 1 (RASSF1, ID: 11186), Ras association domain family member 2 (RASSF2, ID 9770), Ras association domain family member 3 (RASSF3, ID 283349), Ras association domain family member 4 (RASSF4, ID 83937), Ras association domain family member 5 (RASSF5, ID: 83593), Ras association domain family member 7 (RASSF7, ID 8045), Ras association domain family member 8 (RASSF8, ID:11228), Ras association domain family member 10 (RASSF10, ID: 644943), RB transcriptional corepressor 1 (RB1, ID: 5925), retinoic acid receptor beta (RARB, ID: 5915), Rho guanine nucleotide exchange factor 10 (ARHGEF10, ID: 9639), ripply transcriptional repressor 2 (RIPPLY2, ID: 134701), ripply transcriptional repressor3 (RIPPLY3, ID: 53820), secreted frizzled-related protein 1 (SFRP1, ID: 6422), secreted frizzled-related protein 2 (SFRP2, ID: 6423), secreted frizzled-related protein 4 (SFRP4, ID: 6424) secreted frizzled-related protein 5 (SFRP5, ID: 6425), septin 9 (SEPT9, ID: 10801), spectrin repeat containing nuclear envelope protein 1 (SYNEl, ID: 23345), solute carrier family 12 member 8 (SLC12A8, ID: 84561), solute carrier family 16 member 5 (SLC16A5, ID: 9121), SRY-box transcription factor 17 (SOX17, ID: 64321), T-box transcription factor 15 (TBX15, ID: 6913), tissue factor pathway inhibitor 2 (TFPI2, ID: 7980), trafficking regulator and scaffold protein tamalin (TAMALIN, ID: 160622), tropomyosin 4 (TPM4, ID: 7171), TSPY like 5 (TSPYL5, ID: 85453), Scm like with four mbt domains 2 (SFMBT2, ID: 57713), suppressor of cytokine signaling 3 (SOCS3, ID: 9021), suppressor of cytokine signaling 4 (SOCS4, ID: 122809), vav guanine nucleotide exchange factor 3 (VAV3, ID: 10451), Wnt family member 2 (WNT2, ID: 7472), zinc finger protein 304 (ZNF304, ID: 57343), zinc finger protein 568 (ZNF568, ID: 374900), and zinc finger protein 671 (ZNF671, ID: 79891). For the purpose of this application, a CpG island is considered associated with a gene if it is present in the promoter, the body of the gene or up to 50 Kb upstream of the promoter.

The methods of the invention also can be used to evaluate CpG islands associated with the presence or absence of any type of cancer, including specifically those listed herein. The methods of the invention can also be used to evaluate other diseases including infectious, immunological, and neurological diseases.

The methods of the invention are also suitable for evaluating the methylation density of multiple (e.g., 5 or more, 10 or more, 100 or more, 1000 or more, or 10,000 or more) target sequences in samples that include a relatively small amount of DNA or RNA, such as 15 ng or less.

### EXAMPLES

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(29) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

(1) A method for analyzing the methylation status of at least one target sequence in a sample comprising:
(a) providing a sample comprising DNA, wherein the DNA comprises at least one target sequence;
(b) optionally, purifying the DNA from the sample to thereby produce purified DNA;
(c) optionally, ligating a linker to the DNA to thereby produce tagged DNA;
(d) contacting the sample or purified DNA or tagged DNA with at least one methyltransferase that methylates non-cytosine nucleotides; and
(e) optionally, contacting the sample or purified DNA or tagged DNA with at least one methyltransferase that methylates cytosine nucleotides; and
(f) assaying the sample or purified DNA or tagged DNA for the methylation status of one or more cytosine nucleotides in the at least one target sequence.

(2) The method of aspect 1, wherein the methylation status of at least two target sequences are analyzed.

(3) The method of any one of aspects 1-2, wherein the sample or purified DNA or tagged DNA is contacted with at least one methyltransferase that methylates non-cytosine nucleotides and at least one methyltransferase that methylates cytosine nucleotides.

(4) The method of any one of aspects 1-3, wherein the at least one methyltransferase includes at least one methyltransferase that methylates adenine nucleotides present in the at least one target sequence.

(5) The method of any one of aspects 1-4, wherein the at least one methyltransferase includes at least one methyltransferase that methylates adenine residues within a recognition sequence that comprises the sequence AG, RAR, GATC, GTAC, TCGA, CATG, AATT, GAWTC, SATC, AACCA, ACATC, GAATTC, GACGTC, CACAG, or any combination thereof.

(6) The method of any one of aspects 1-5, wherein the at least one methyltransferase includes at least one methyltransferase that methylates adenine nucleotides without a specific recognition sequence.

(7) The method of any one of aspects 1-6, wherein the at least one methyltransferase is M.EcoKDam, M.CviQ1, M.CviQX1, M.CvQII, M.TaqI, M.Tsp509I, M.AatII, M.BceJI, M.EcoR1, M.EcoGII or any combination thereof.

(8) The method of any one of aspects 1-7, wherein the at least one methyltransferase methylates at least 0.3% of nucleotides in the sample.

(9) The method of any one of aspects 1-8, wherein the at least one methyltransferase methylates at least 0.8% of nucleotides in the sample.

(10) A method for analyzing the methylation status of at least one target sequence present in a sample comprising:
(a) providing a sample comprising DNA, wherein the DNA comprises at least one target sequence;
(b) optionally, purifying the DNA from the sample to thereby produce purified DNA;
(c) optionally, ligating a linker to the DNA to thereby produce tagged DNA;
(d) contacting the sample or purified DNA or tagged DNA with at least one methyltransferase, wherein the at least one methyltransferase methylates at least 0.7% of cytosine nucleotides in the sample; and
(e) assaying the sample or purified DNA or tagged DNA for the methylation status of one or more cytosine nucleotides in the at least one target sequence.

(11) The method of any one of aspects 1-10, wherein the at least one methyltransferase methylates at least 5% of nucleotides in the sample.

(12) The method of aspect 10 or 11, wherein the cytosine methyltransferase methylates cytosine residues within a recognition sequence that comprises the sequence AGCT, GGCC, GCGC, GTAC, GATC, TCGA, CCGG, GCNGC, CCWGG, RCATGY, GAGCTC, RGCB, CCD, CGR, GC, CC, or any combination thereof.

(13) The method of any one of aspects 10-13, wherein the at least one methyltransferase is M.Alu, M.HaeIII, M.HhaI, M.RsaI, M.Sau3AI, M.EsaLHCI, M.EsaBC2I, M.HpaII, M.MspI, M.EcoKDcm, M.BamHI, M.CviPI, M.CviPII, M.CviQIX, M.CviQVIII, M.CviQX or any combination thereof.

(14) A method for quantitating the methylation status of at least one target sequence present in a sample to quantitate the percentage of the at least one target sequence that is methylated, comprising the method of any one of aspects 1-14, and further comprising:
(g) comparing the amount of methylated and unmethylated cytosine nucleotides in the at least one target sequence to a corresponding amount in a standard.

(15) The method of aspect 14, wherein the standard is generated from known amounts of methylated and unmethylated DNA.

(16) A method for analyzing the density of methylation of at least one target sequence present in a sample to improve the quantitation of the methylation of two or more cytosines within the at least one target sequence comprising the method of aspect 15 or 16, wherein the methylation status of two or more cytosine nucleotides in the at least one target sequence is determined during the assay step.

(17) The method of any one of aspects 1-16, wherein the assay step comprises digesting the sample or purified DNA or tagged DNA with a sequence specific restriction endonuclease.

(18) The method of any one of aspects 1-17, wherein the assay step comprises targeted sequencing, next generation sequencing or direct sequencing.

(19) The method of any one of aspects 1-18, wherein the assay step comprises using single nucleotide primer extension, fluorescent-based quantitative PCR, headloop suppression PCR, ligation-mediated amplification, microarray analysis, bead hybridization, flow cytometry, mass spectrometry, or any combination thereof.

(20) The method of any one of aspects 1-19, wherein the assaying step comprises using a bisulfite salt or enzymes to convert unmodified cytosine nucleotides into uracil nucleotides.

(21) The method of any one of aspects 1-20, wherein the sample is a tissue obtained during a biopsy or a surgical resection.

(22) The method of any one of aspects 1-21, wherein the sample is a bodily fluid.

(23) The method of aspect 22, wherein the bodily fluid is blood, blood plasma, blood serum, urine, sputum, ejaculate, semen, prostatic fluid, tears, sweat, saliva, lymph fluid, bronchial lavage, pleural effusion, peritoneal fluid, meningeal fluid, amniotic fluid, glandular fluid, fine needle aspirates, nipple aspirate fluid, spinal fluid, conjunctival fluid, vaginal fluid, duodenal juice, pancreatic juice, pancreatic ductal epithelium, pancreatic tissue bile, cerebrospinal fluid, or any combination thereof.

(24) The method of any one of aspects 1-23, wherein the sample comprises fragments of fewer than 150 contiguous nucleotides, wherein the fragments include the at least one target sequence.

(25) The method of any one of aspects 1-24, wherein the sample includes less than 15 ng of DNA.

(26) A method for analyzing the methylation status of at least one target sequence present in a sample comprising:
(a) providing a sample comprising RNA, wherein the RNA comprises at least one target sequence;
(b) optionally, purifying the RNA from the sample to thereby produce purified RNA;
(c) optionally, ligating a linker to the RNA to thereby produce tagged RNA;
(d) contacting the sample or purified RNA or tagged RNA with at least one methyltransferase that methylates non-cytosine nucleotides; and
(e) assaying the sample or purified RNA or tagged RNA for the methylation status of one or more cytosine nucleotides in the at least one target sequence.

For the following examples, the deamination temperature was set at 70° C. and the amount of DNA, the concentration of bisulfite salts, and the length of treatment were each varied. The experiments were performed using DNA from a prostate cancer tissue sample (D1b) or a leukemia cancer cell line, (CCL-119 (119), ATCC cat #CRL-2264). The MS-qPCR reactions were performed on the equivalent of 1 ng of DNA (pre-bisulfite) unless stated otherwise. Assays were designed for various CpG islands and were named for the purpose of the examples after the nearest gene on the chromosome. If the assay name was followed by an “rc”, it indicates that the probes and primers were designed from the reverse complement of the CpG island sequence (i.e. reverse strand). The DNA methyltransferases used in the various examples were: AluI (a), Dam (D), HaeIII (h), HhaI (H), MspI (M), and EcoGII (E).

### Example 1

This example demonstrates the detection of methylation of CpG islands associated with 13 genes, ADCY4 (SEQ ID NO: 1), AOX1 (SEQ ID NO: 2), CYBA (SEQ ID NO: 3), EPHX3 (SEQ ID NO: 4), GPR62 (SEQ ID NO: 5), HOXA5 (SEQ ID NO: 8), HOXA7 (SEQ ID NO: 9), HOXD3b (SEQ ID NO: 12), HOXD3c (SEQ ID NO: 13), HOXD9 (SEQ ID NO: 15), KLK10 (SEQ ID NO: 16), NODAL (SEQ ID NO: 18), and RASSF1 (SEQ ID NO: 19) from untreated (no added methylation) and in vitro methylated prostate cancer tumor DNA (D1b). The DNA was methylated with the EcoGII methyltransferase which introduces methyl groups on >50% of adenine residues (per manufacturer information). The sequence of the CpG islands associated with the selected genes is provided in the sequence listing. The primers and probes were designed to complement either the forward or the reverse strand (rc) of the target sequence after bisulfite treatment. The methylation of the cancer DNA (D1b) at the selected target sequences was previously determined.

D1b tumor DNA was isolated from formalin-fixed paraffin embedded prostatectomy tissues as previously described (Brikun et al., Biomark. Res. 2(1): 25 (2014)) and quantitated using the Invitrogen Quant-IT ds DNA HS (Thermofisher cat #33232). Up to 0.5 μg was methylated using EcoGII methyltransferase (New England Biolabs “NEB” Beverly, MA) according to supplier's recommendations. Fifteen nanograms of unmethylated or EcoGII-methylated D1b DNA were deaminated as follows: the DNA was denatured twice, first by incubation at 95° C. for 5 minutes then placed on ice for 5 minutes followed by denaturation in 0.2M NaOH at 44° C. for 12 minutes. Two hundred microliters of a bisulfite solution (700 milligrams of Ammonium sulfite [Sigma-Aldrich cat #358983] and 400 milligrams of sodium metabisulfite (Sigma-Aldrich cat #13459) were dissolved in a 3.4 ml 50% ammonium hydrogen sulfite [Wako cat #013-23931]) were added and the DNA was incubated at 70° C. for 3 hours. The DNA was then diluted with water to 500 μl and concentrated over a 0.5 ml Amicon Ultra-0.5 50 kD centrifugal filter unit (Millipore Sigma cat #UFC505024) (5 min spin at 10000 rpm) and washed once with 400 μl of H₂O. It was then desulfonated on the Amicon column by adding 300 μl of 0.33 mM NaOH and incubating at room temperature for 21 min. After washing the column with 3×300 μl of H₂O, the DNA was recovered in 60 μl of H₂O and stored at −20° C.

Methylation-specific PCR (MS-qPCR): Probes and primers were purchased from Biosearch Technologies, Novato, CA or Eurofins MWG Operon, Huntsville, AL. All probes were labeled with FAM (5′) and BHQ1 (3″) quencher. Markers were amplified individually. Each reaction was carried out in 20 μl of 1× TaKaRa HotStart Taq DNA polymerase buffer (10 mM Tris-HCl pH 8.3, 50 mM KCl, 1.5 mM MgCl₂) supplemented with 1.0 mM magnesium chloride, 0.20 mM dNTPs, 0.5 μM forward primer (same orientation as the probe), 1.0 μM reverse primer, 1.25 μM probe, 0.5 units of TaKaRa HotStart Taq DNA polymerase (Takara cat #R007, Ann Arbor, MI) and 4 μl of bisulfite-treated DNA. The DNA input in each MS-PCR reaction is equivalent to 1 ng of D1b DNA prior to bisulfite treatment. MS-qPCR reactions were performed on an ABI QuantStudio 6 real time PCR instrument for 50 cycles of 95° C. for 15 seconds, 68° C. for 20 seconds, and 64° C. for 20 seconds after a 5 min denaturation at 95° C.

The primers and probes used to amplify each marker are: ADCY4 (SEQ ID NOs: 22, 23, 24), AOX1 (SEQ ID NOs: 25, 26, 27), CYBA (SEQ ID NOs: 28, 29, 30), EPHX3 (SEQ ID NOs: 31, 32, 33), GPR62 (SEQ ID NOs: 34, 35, 36), HOXA5 (SEQ ID NOs: 37, 38, 39), HOXA7 (SEQ ID NOs: 40, 41, 42), HOXD3b (SEQ ID NOs: 43, 44, 45), HOXD3c (SEQ ID NOs: 46, 47, 48), HOXD9 (SEQ ID NOs: 49, 50, 51), KLK10 (SEQ ID NOs: 52, 53, 54) NODAL (SEQ ID Nos: 55, 56, 57), NODALrc (SEQ ID NOs: 58, 59, 60), and RASSF1 (SEQ ID NOs: 61, 62, 63).

Table 2 shows the Cq values obtained with MS-qPCR reactions of D1b and D1b methylated with EcoGII methyltransferase. For the purpose of this application, Cq (quantification cycle) and Ct (threshold cycle, reported by the QuantStudio™ 6 real-time PCR instrument) are synonymous and indicate the fractional number of cycles needed for the fluorescent signals to rise above the background fluorescence detected by the instrument. A lower Cq number generally indicates a higher number of target sequences in the sample. The MS-qPCR reactions yield a positive signal (i.e. a Cq value) when targets are methylated at the CpG dinucleotides present within the primers and probes. Some of the targets such as ADCY4, GPR62, HOXD3b could be detected from both unmethylated and EcoGII methylated D1b DNA while others such as AOX1, CYBA, HOXA7, KLK10 were only recovered from EcoGII methylated DNA under the analytical conditions used for this example.

TABLE 2

| | Sample Name | | | | | |
|---|---|---|---|---|---|---|
| Marker | D1b_U1 | D1b_U2 | D1b_U3 | D1b_E1 | D1b_E2 | D1b_E3 |
| ADCY4 | — | 37.84 | 39.25 | 38.00 | 37.20 | 38.00 |
| GPR62 | 37.74 | — | 38.77 | 35.64 | 37.21 | 36.01 |
| NODAL | 38.30 | 38.60 | — | 36.39 | 35.02 | 37.68 |
| HOXA5 | — | 39.67 | — | 37.84 | 37.91 | 37.55 |
| HOXD3b | 38.84 | — | — | 36.90 | 36.65 | 35.50 |
| HOXD9rc | — | 39.84 | — | 37.30 | 36.55 | 35.94 |
| EPHX3 | — | 42.39 | — | 37.20 | 47.31 | 36.66 |
| AOX1rc | — | — | — | 36.51 | 37.06 | 36.21 |
| CYBA | — | — | — | 38.32 | 38.42 | 38.01 |
| NODALrc | — | — | — | 36.45 | 36.67 | 36.93 |
| RASSF1 | — | — | — | 38.30 | 39.07 | 39.92 |
| HOXD3c | — | — | — | 38.97 | 38.50 | 35.71 |
| HOXA7 | — | — | — | 37.22 | 37.55 | 38.31 |
| KLK10 | — | — | — | 38.18 | 39.55 | 43.43 |

Table 2 shows the Cq values generated from the MS-qPCR reactions from D1b unmethylated (D1b-U) or EcoGII methylated D1b DNA (D1b-E). Three replicas labeled as 1, 2 or 3 were performed for each marker. A dash (-) indicates that no signal was detected above background.

This example shows that in vitro methylation at adenine residues prior to bisulfite treatment improved the detection of multiple markers from small amounts of DNA. Without the additional methylation, some markers such as KLK10, HOXA7, HOXD3c could not be detected while others such as ADCY4, GPR62 and NODAL could. Furthermore, the EcoGII-methylated DNA resulted in a more robust and reproducible amplification for all markers analyzed. This example shows that in vitro methylation at adenine residues improved the recovery of the methylation signature of the D1b tumor DNA.

### Example 2

This example demonstrates the detection of methylation of CpG islands associated with 9 genes, GPR62 (SEQ ID NO: 5), HOXA5 (SEQ ID NO: 8), HOXA11 as (SEQ ID NO: 10), HOXD3 (SEQ ID NOs: 12, 13), HOXD4 (SEQ ID NO: 14), KLK10 (SEQ ID NO: 16), NODAL (SEQ ID NO: 18), RIPPLY (SEQ ID NO: 20), and SEPT9 (SEQ ID NO: 21) from untreated (no added methylation) and in vitro methylated CCL-119 leukemia cell line DNA. The sequence of the additional CpG islands is provided in the sequence listing.

Up to 1 μg 119 DNA was methylated with 3 methyltransferases AluI, HhaI and EcoGII (aHE) in 1× CutSmart buffer according to manufacturer's recommendations. Fifteen nanograms of untreated 119 or aHE methylated 119 DNA were analyzed as described in Example 1 except the length of the treatment was extended to 4 hours and the equivalent of 0.5 nanogram of 119 DNA (pre-bisulfite) was used for the MS-qPCR reactions. Two reactions were performed on each DNA.

In addition to the oligonucleotides listed in Example 1, the following probes and primers were used: HOXA11 as (SEQ ID NOs: 64, 65, 66), HOXD4 (SEQ ID NOs: 67, 68, 69), RIPPLY2 (SEQ ID NOs: 70,71,72), and SEPT9 (SEQ ID NOs: 73,74,75).

TABLE 3

| | Sample | | | |
|---|---|---|---|---|
| Marker | 119_1 | 119_2 | 119aHE_1 | 119aHE_2 |
| GPR62 | — | 39.63 | 36.28 | 35.40 |
| HOXA5 | — | — | 38.63 | 42.88 |
| HOXA11as | — | 38.08 | 38.14 | 38.33 |
| HOXD3b | — | — | 39.70 | 38.70 |
| HOXD3c | — | — | 43.93 | 38.39 |
| HOXD4rc | 44.03 | — | 37.52 | 39.56 |
| KLK10 | — | — | 38.49 | 37.78 |
| NODAL | — | 39.64 | 37.39 | 37.52 |
| NODALrc | — | — | 41.87 | 42.90 |
| RIPPLY2 | — | 36.85 | 35.78 | 42.82 |
| SEPT9rc | — | — | 36.45 | 35.24 |

Table 3 shows the Cq values obtained from 2 MS-qPCR reactions from unmethylated (119) or aHE methylated 119 DNA (119-aHE). A dash (-) indicates that no signal was detected above background.

This example shows improved detection of multiple markers when DNA is methylated at AluI, HhaI and EcoGII sites. The aHE-methylated DNA shows a more reliable amplification than the unmethylated DNA for all markers analyzed. Multiple markers such as KLK10, HOXD3b, HOXD3c and SEPT9rc couldn't be detected without the in vitro methylation. This example shows that the additional in vitro methylation at cytosine and adenine residues improved the recovery of the methylation signature of the 119 tumor cell line DNA.

### Example 3

This example compares 119 DNA methylated with AluI and HaeIII methyltransferases to DNA methylated with AluI, HaeIII, and EcoGII methyltransferases. Fifteen CpG islands associated with 15 genes which include genes from Example 1 and 2 in addition to HOXA1, HOXCas1 and NEUROG3 were analyzed. Fifty nanograms of DNA were analyzed as described in example 1 and 2 except that the bisulfite solution was prepared by dissolving 3.5 g Ammonium sulfite [Sigma-Aldrich cat #358983] in a final volume of 10 ml 50% ammonium hydrogen sulfite [Wako cat #013-23931]), and the treatment was performed for 5 hours. The equivalent of 1 ng of DNA pre-bisulfite was used for the MS-qPCR reactions. The bisulfite reactions were performed in 10 replicas (labeled 1 to 10). The probes and primers used to detect HOXA1, HOXCas1 and NEUROG3 were: HOXA1 (SEQ ID NOs: 76, 77, 78), HOXCas1 (SEQ ID NOs: 79, 80, 81) and NEUROG3 (SEQ ID NOs: 82, 83, 84). The results show improved marker detection when greater than 0.7% of nucleotides are methylated.

TABLE 4

| | Marker | | | | | | | |
|---|---|---|---|---|---|---|---|---|
| Sample | AOX1 | GPR62 | KLK10 | HOXD9 | NODAL | NEUROG3 | RIPPLY2 | HOXA5 |
| 119ah_1 | — | — | 46.9 | — | — | 38.6 | — | — |
| 119ah_2 | — | — | 39.3 | — | — | — | — | — |
| 119ah_3 | — | — | — | — | — | — | — | — |
| 119ah_4 | — | — | — | — | — | — | — | — |
| 119ah_5 | 38.1 | — | — | — | — | — | — | — |
| 119ah_6 | — | — | 40.4 | — | 40.7 | 42.5 | — | — |
| 119ah_7 | — | — | — | — | — | — | — | — |
| 119ah_8 | — | — | — | — | — | — | — | — |
| 119ah_9 | — | — | — | — | — | — | — | — |
| 119ah_10 | — | — | — | — | — | — | 36.7 | — |
| 119ahE_1 | 35.5 | 37.5 | 37.7 | — | 41.8 | 37.8 | 34.1 | — |
| 119ahE_2 | 34.7 | 37.7 | 38.4 | — | 37.7 | 36.0 | 34.8 | 37.0 |
| 119ahE_3 | 35.3 | 37.6 | 37.9 | 35.5 | 36.8 | 35.4 | 36.5 | 40.4 |
| 119ahE_4 | 34.3 | 36.0 | 38.0 | 37.1 | 37.8 | 36.4 | 36.6 | 36.8 |
| 119ahE_5 | 41.9 | 36.2 | 41.0 | 35.8 | 39.8 | 34.2 | 34.8 | 37.9 |
| 119ahE_6 | 35.6 | 36.9 | 38.5 | 38.0 | — | 37.5 | 35.5 | — |
| 119ahE_7 | 35.7 | 36.3 | 37.6 | 35.5 | 37.8 | 35.1 | 34.2 | 35.4 |
| 119ahE_8 | 35.1 | 36.0 | 40.2 | 40.2 | 38.6 | 38.7 | 35.3 | 40.0 |
| 119ahE_9 | 35.9 | — | 42.2 | 37.8 | 43.2 | 34.6 | 34.9 | 35.6 |
| 119ahE_10 | 35.4 | 35.9 | 37.1 | 36.2 | 37.8 | 35.4 | 34.4 | 39.1 |

| | Marker | | | | | | |
|---|---|---|---|---|---|---|---|
| Sample | HOXA1 | HOXD4rc | HOXA11as | HOXD3b | HOXD3c | HOXCas1 | SEPT9 |
| 119ah_1 | — | — | — | — | — | — | — |
| 119ah_2 | — | — | — | — | — | — | — |
| 119ah_3 | — | 40.2 | — | — | — | — | — |
| 119ah_4 | — | — | — | — | — | — | — |
| 119ah_5 | — | — | — | — | — | — | — |
| 119ah_6 | — | — | — | — | — | — | — |
| 119ah_7 | — | — | — | — | — | — | — |
| 119ah_8 | — | 42.5 | — | — | — | — | — |
| 119ah_9 | — | — | — | — | — | — | 37.4 |
| 119ah_10 | — | — | — | — | — | — | — |
| 119ahE_1 | 34.9 | — | 36.4 | 37.4 | 35.9 | 35.3 | — |
| 119ahE_2 | 36.6 | 40.1 | 35.6 | 37.7 | 36.9 | 42.3 | 35.9 |
| 119ahE_3 | 35.2 | 39.5 | 36.0 | 36.9 | 36.9 | 37.9 | 35.2 |
| 119ahE_4 | 36.2 | 40.3 | 35.9 | 38.0 | — | 35.3 | 35.0 |
| 119ahE_5 | 34.9 | 42.7 | 35.0 | 39.4 | 39.1 | 39.0 | 35.2 |
| 119ahE_6 | 35.1 | 45.0 | 38.2 | 37.1 | 38.6 | 37.0 | — |
| 119ahE_7 | 36.1 | 36.9 | 36.1 | 38.0 | 37.4 | 36.5 | 33.5 |
| 119ahE_8 | 34.4 | 40.2 | 36.1 | 36.3 | 37.0 | 43.0 | 33.0 |
| 119ahE_9 | 35.0 | 38.7 | 35.7 | 37.0 | 36.8 | 36.9 | 34.2 |
| 119ahE_10 | — | 38.5 | 37.0 | — | 39.8 | — | 35.3 |

Table 4 shows the Cq values obtained from MS-qPCR reactions from 119 DNA methylated with AluI and HaeIII methyltransferases (119ah) or AluI, HaeIII and EcoGII methyltransferases (119-ahE). A dash (-) indicates that no signal was detected above background.

### Example 4

This example demonstrates the detection of methylation in 7 CpG islands associated with 6 genes, GPR62, HOXD3, NEUROG3, HIF3a, RIPPLY2 and SEPT9 from 119 DNA methylated in vitro using different combinations of enzymes. It shows that increasing methylation within CpG islands beyond the AluI and HaeIII sites improves the recovery of markers. The 119 genomic DNA was methylated sequentially with various methyltransferases (AluI, HaeIII, Dam, HhaI, MspI and EcoGII) according to manufacturer's recommendations. Fifteen nanograms of each DNA were deaminated in duplicate as described in Example 3. The markers were amplified in duplicates from 2 bisulfite reactions (la, lb, and 2a, 2b) using MS-qPCR assays as described in Example 1. The primers and probes were as listed in previous examples. For HIF3a, the primers and probe were SEQ ID NOs: 85, 86, 87, and 88. The results (Cq values) are shown in Table 5.

TABLE 5

| Sample + | | Marker | | | | | | |
|---|---|---|---|---|---|---|---|---|
| MTs used | DNA | GPR62 | HIF3a | NEUROG3 | RIPPLY2 | SEPT9rc | HOXD3c | HOXD3b |
| 119ah | 1a | — | — | — | — | — | — | — |
| | 1b | — | — | — | — | — | — | — |
| | 2a | — | — | — | — | — | — | — |
| | 2b | — | 38.34 | — | — | — | — | — |
| 119ahDHM | 1a | 36.18 | 35.33 | 34.97 | 34.06 | 34.97 | 37.61 | 37.87 |
| | 1b | 34.24 | 33.74 | 34.85 | 34.63 | 33.95 | 35.77 | 36.15 |
| | 2a | 36.87 | 37.88 | 34.96 | 34.42 | 35.19 | 36.76 | 39.63 |
| | 2b | 36.71 | 38.55 | 35.89 | 34.74 | 34.47 | 37.02 | 37.63 |
| 119ahDHE | 1a | 34.44 | 34.28 | 35.13 | 33.22 | 35.50 | 35.62 | 35.48 |
| | 1b | 36.00 | 34.41 | 34.95 | 33.73 | 34.54 | 36.43 | 36.01 |
| | 2a | 34.80 | 39.06 | 34.75 | 38.83 | 33.82 | 37.60 | 36.78 |
| | 2b | 35.77 | 48.73 | 34.69 | 35.20 | 33.95 | 35.21 | 39.63 |
| 119E | 1la | 39.99 | 35.52 | 36.84 | 33.77 | 36.08 | 37.09 | 37.06 |
| | 1b | 37.24 | 36.34 | 35.91 | 33.26 | 36.37 | 35.06 | 38.68 |
| | 2a | 36.98 | 38.58 | 36.20 | 34.51 | 33.49 | 36.95 | 36.39 |
| | 2b | 37.87 | 39.13 | 34.75 | 34.48 | 33.77 | 34.83 | 37.44 |
| 119aHE | 1a | 36.61 | 36.66 | 36.16 | 34.62 | 34.71 | 43.73 | 37.99 |
| | 1b | 37.23 | 35.79 | 36.38 | 34.78 | 34.73 | 36.03 | 35.94 |
| | 2a | 36.33 | 38.05 | 34.68 | 35.25 | 33.53 | 37.10 | 36.68 |
| | 2b | 36.82 | 44.44 | 34.58 | 34.01 | 33.61 | 36.43 | 38.25 |

Table 5 shows the Cq values obtained from MS-qPCR reactions of 119 DNA methylated with AluI and HaeIII (119ah), AluI, HaeIII, Dam, HhaI and MspI (119ahDHM), AluI, HaeIII, Dam, HhaI and EcoGII (119ahDHE), EcoGII (119E), AluI, HaeIII, EcoGII (119ahE). A dash (-) indicates that no signal was detected above background.

This example shows that increasing the methylation of CpG islands improves the recovery of markers and that it can be accomplished using various combinations of methyltransferases that modify adenine and cytosine residues.

### Example 5

This example demonstrates the detection of methylation of 6 CpG islands associated with 6 genes, AOX1, HOXA1, HOXD3c, HOXD9, NEUROG3, and RIPPLY2 from 15 ng of white blood cell DNA spiked with increasing amounts of 119 DNA. Both DNAs were methylated in vitro with AluI, HhaI and EcoGII (aHE) methyltransferases as described in Example 2. Two hundred and fifty picograms, 0.5 ng, 1 ng, 2 ng or 4 ng of 119 aHE DNA were added to 15 ng of aHE methylated DNA isolated from white blood cells. The bisulfite reactions were performed as described in Example 3 except the length of treatment was 75 min.

The markers were detected using a nested PCR strategy. First, all six markers were preamplified as a multiplex for a limited number of cycles with primers that amplify both methylated and unmethylated templates. The primers used were as follows: AOX1rc (SEQ ID Nos: 89, 90), HOXA1 (SEQ ID Nos: 91, 92), HOXD3c (SEQ ID Nos: 93, 94), HOXD9rc (SEQ ID Nos: 95, 96), NEUROG3 (SEQ ID Nos: 97, 98), and RIPPLY2 (SEQ ID Nos: 99, 100). Second, markers were detected individually from the preamplification reactions using primers and probes specific to the methylated templates and the results were tabulated in Table 6.

The preamplification reactions were performed on one quarter of the bisulfite treated DNA in duplicate as follows: following a 5 min denaturation at 95° C., primary PCR reactions were performed for 15 cycles of 95° C. for 15 seconds, 58° C. for 40 seconds, and 72° C. for 20 seconds using an Eppendorf mastercycler in a 30 microliter reaction of 1× TaKaRa EpiTaq HS DNA polymerase buffer supplemented with 1.0 mM magnesium chloride, 0.20 mM dNTPs, 0.2 μM of each primer (SEQ ID NOs: 89-100) and 0.5 units each of TaKara EpiTaq HS (Takara cat #R110A and TaKaRa HotStart Taq DNA polymerase (Takara cat #R007). For the 0.25 ng input, the primary amplifications are expected to contain less than 10 copies of the methylated targets assuming that the methylation of all 119 cancer cells is uniform.

Following the primary amplification, the DNA was diluted to a final volume of 150 microliter with water and four microliters were used for target detection. The nested primers used for each marker were as described in previous examples except for AOX1 (SEQ ID NOs: 25, 26, and 101) and NEUROG3 (SEQ ID NOs: 102, 103, and 104).

TABLE 6

| | Marker | | | | | |
|---|---|---|---|---|---|---|
| Sample | HOXA1 | HOXD3c | AOX1rc | HOXD9rc | NEUROG3 | RIPPLY2 |
| 0.0 ng 119aHE-a | — | — | — | — | — | — |
| 0.0 ng 119aHE-b | — | — | — | — | — | — |
| 0.25 ng 119aHE-a | 29.05 | — | 29.43 | 32.72 | — | 29.51 |
| 0.25 ng 119aHE-b | — | — | 29.46 | 32.91 | — | 29.35 |
| 0.5 ng 119aHE-a | 29.07 | 31.21 | 28.87 | 29.53 | 33.32 | 29.61 |
| 0.5 ng 119aHE-b | — | — | 29.08 | 29.45 | 32.96 | 29.24 |
| 1.0 ng 119aHE-a | 28.38 | 33.54 | 27.30 | 27.43 | 28.40 | 26.81 |
| 1.0 ng 119aHE-b | 27.91 | 32.95 | 38.27 | 30.21 | 28.10 | 27.30 |
| 2.0 ng 119aHE-a | 27.27 | 30.07 | 27.62 | 28.00 | 27.11 | 28.41 |
| 2.0 ng 119aHE-b | 27.42 | 31.76 | 28.18 | 30.04 | 26.88 | 27.62 |
| 4.0 ng 119aHE-a | 25.43 | 30.22 | 25.57 | 27.90 | 26.28 | 25.50 |
| 4.0 ng 119aHE-b | 25.80 | 29.39 | 27.05 | 27.78 | 26.39 | 26.90 |

Table 6 shows the Cq values obtained from MS-qPCR reactions of 15 ng of WBC-aHE spiked with increasing amounts of 119-aHE DNA. A dash (-) indicates that no signal was detected above background. Markers were amplified in duplicates from each DNA (labeled “a” and “b”).

This example shows that markers can be detected from minimal amounts of tumor cell line DNA in a background of excess normal human DNA.

### Example 6

This example demonstrates the detection of methylation of 10 CpG islands associated with 9 genes, from 15 ng of D1b DNA methylated with AluI/HaeIII or AluI/HaeIII/HhaI methyltransferases as described in Example 3. Methylation with AluI and HhaI methyltransferases was performed concurrently using the AluI methylation buffer. The bisulfite reactions were performed as described in Example 1 except the length of treatment was extended to 3 hours and 45 min. The MS-qPCR assays to detect various markers were as described in Examples 1 and 2.

TABLE 7

| | Sample | | | | | |
|---|---|---|---|---|---|---|
| Marker | D1b_ah | D1b_ah | D1b_ah | D1b_ahH | D1b_ahH | D1b_ahH |
| ADCY4 | — | — | — | 38.19 | 44.60 | 36.93 |
| EPHX3 | — | — | — | — | 37.34 | 37.76 |
| GPR62 | — | — | — | 37.20 | 36.96 | 40.94 |
| HOXA5 | — | — | — | 35.87 | 39.23 | 36.69 |
| HOXA7 | — | — | 40.91 | — | 36.14 | 36.95 |
| HOXD3b | — | — | — | 37.29 | 37.77 | 37.94 |
| HOXD3c | — | — | — | 40.83 | — | 37.30 |
| HOXD9rc | — | — | 40.23 | 36.06 | 38.10 | 36.66 |
| NODAL | — | 39.83 | — | 37.77 | 39.08 | 38.81 |
| NODALrc | — | — | — | 37.11 | 40.27 | — |

This example shows that adding methylation at HhaI sites to AluI and HaeIII methylated DNA enables the detection of markers when DNA undergoes longer bisulfite treatment.

### Example 7

This example demonstrates the detection of methylation of CpG islands associated with 10 genes, AOX1 (SEQ ID NO: 2), EPHX3 (SEQ ID NO: 4), GPR62 (SEQ ID NO: 5), HOXA7 (SEQ ID NO: 9), HOXD3b (SEQ ID NO: 12), HOXD3c (SEQ ID NO: 13), HOXD4 (SEQ ID NO: 14), HOXD9 (SEQ ID NO: 15), NEUROG3 (SEQ ID NO: 17) and NODAL (SEQ ID NO: 18) from untreated (no added methylation) and in vitro methylated D1b prostate tumor DNA. The DNA was methylated with the AluI, HhaI and EcoGII methyltransferases. The sequence of the CpG islands associated with the selected genes is provided in the sequence listing. The primers and probes were designed to complement either the forward or the reverse strand (rc) of the target sequence after bisulfite treatment. The methylation of the prostate cancer DNA (D1b) at the selected target sequences was previously determined.

Up to 0.5 μg was methylated using AluI, HhaI, and EcoGII (aHE) methyltransferases (New England Biolabs “NEB” Beverly, MA) as described in Example 2. Fifteen nanograms of unmethylated or aHE-methylated D1b DNA were deaminated as described in Example 1. Four replicas labeled 1 through 4 were performed for each DNA.

The markers were detected using a nested PCR strategy. First, the bisulfite treated DNAs were amplified with 2 sets of markers as described in Example 5 except that the DNA input for the multiplex reactions was equivalent to 3 ng of pre-bisulfite DNA and 18 cycles were performed instead of 15. The first multiplex primer mix included AOX1 (SEQ ID NOs: 89 and 90), GPR62 (SEQ ID NOs: 105 and 106), EPHX3 (SEQ ID NOs: 107 and 108), HOXD3c (SEQ ID NOs: 93 and 94), HOXD9rc (SEQ ID NOs: 95 and 96), and NODALrc (SEQ ID NOs: 117 and 118). The second multiplex primer mix included HOXD4rc (SEQ ID NOs: 113 and 114), HOXA7 (SEQ ID NOs: 109 and 110), HOXD3b (SEQ ID NOs: 111 and 112), NEUROG3 (SEQ ID NOs: 97 and 98) and NODAL (SEQ ID NOs: 115 and 116). Second, markers were detected individually from the preamplification reactions using primers and probes specific to the methylated templates and the results were tabulated in Table 8.

Following the primary amplification, the DNA was diluted with water to a final volume of 450 microliter and four microliters were used for target detection. The PCR reactions were performed as described in Example 1. The nested primers used for each marker were as follows: AOX1 (SEQ ID NOs: 25, 26, and 101), GPR62 (SEQ ID NOs: 34, 35, 120), EPHX3 (SEQ ID NOs: 31, 32, 33), HOXD3c (SEQ ID NOs: 46, 47, 119), HOXD9rc (SEQ ID NOs: 49, 50, 51), NODALrc (SEQ ID NOs: 58, 59, 60) HOXD4rc (SEQ ID NOs: 67, 68, 69), HOXA7 (SEQ ID NOs: 40, 41, 42), HOXD3b (SEQ ID NOs: 43, 44, 45), NEUROG3 (SEQ ID NOs: 102, 103, 104) and NODAL (SEQ ID NOs: 55, 56, 57). Markers were amplified in duplicates from each bisulfite-treated DNA (labeled a and b).

TABLE 8

| | Sample | | | | | | | | | |
|---|---|---|---|---|---|---|---|---|---|---|
| [symbol] arker | D1b_1a | D1b_1b | D1b_2a | D1b_2b | D1b_3a | D1b-3b | D1b-4a | D1b-4b | D1b_ahH-1a | D1b_ahH-1b |
| [symbol] OX1rc | — | — | — | — | — | — | 31.22 | 31.45 | 30.61 | 30.79 |
| [symbol] PR62 | — | — | 35.27 | 32.92 | — | 39.3 | 28.22 | 26.62 | 43.82 | 35.79 |
| [symbol] PHX3 | — | — | — | — | — | — | 38.86 | — | 28.86 | 28.8 |
| [symbol] OXA7 | — | — | 34.33 | 33.65 | 29.92 | 28.99 | 28.37 | 27.98 | 27.06 | 26.4 |
| [symbol] XD4rc | 27.71 | 27.9 | 36.18 | 35.36 | 36.72 | 36.91 | 29.15 | 29.83 | 30.77 | 31.42 |
| [symbol] XD9rc | 29.92 | 30.01 | 31.22 | 31.27 | 40.13 | 36.54 | 29.08 | 29 | 25.77 | 25.85 |
| [symbol] XD3b | — | 40.66 | 36.09 | 34.78 | 38.1 | 36.96 | 37.22 | 39.71 | 32.85 | 32.81 |
| [symbol] XD3c | 29.08 | 30.25 | 29.09 | 30.67 | 35.63 | 39.65 | 39.9 | — | 27.32 | 28.67 |
| [symbol] UROG3 | — | — | — | — | — | — | — | — | 27.96 | 27.71 |
| [symbol] DALrc | — | — | 31.92 | 31.4 | 43.36 | 40.26 | — | — | 29.96 | 29.69 |
| [symbol] ODAL | 26.57 | 27.72 | 27.34 | 28.12 | 27.04 | 28.11 | 28.95 | 27.9 | 27.23 | 25.21 |

| | Sample | | | | | |
|---|---|---|---|---|---|---|
| [symbol] arker | D1b_ahH-2a | D1b_ahH-2b | D1b_ahH-3a | D1b_ahH-3b | D1b_ahH-4a | D [symbol] h [symbol] |
| [symbol] OX1rc | 28.16 | 28.09 | 27.84 | 27.74 | 27.21 | 2 [symbol] |
| [symbol] PR62 | 27.15 | 25.79 | 27.48 | 25.94 | 28.85 | 2 [symbol] |
| [symbol] PHX3 | 30.58 | 30.81 | 28.85 | 28.97 | 26.3 | 2 [symbol] |
| [symbol] OXA7 | 27.35 | 26.66 | 27.73 | 26.64 | 26.96 | 2 [symbol] |
| [symbol] XD4rc | 25.84 | 26.11 | 29.1 | 29.33 | 25.76 | 2 [symbol] |
| [symbol] XD9rc | 26.67 | 26.66 | 26.26 | 26.35 | 26.43 | 2 [symbol] |
| [symbol] XD3b | 34.58 | 34.27 | 32.81 | 34.74 | 32.6 | 3 [symbol] |
| [symbol] XD3c | 25.67 | 26.94 | 25.95 | 27.31 | 25.53 | 2 [symbol] |
| [symbol] UROG3 | 29.92 | 29.04 | 25.59 | 25.4 | 27.35 | 2 [symbol] |
| [symbol] DALrc | 26.53 | 26.48 | 27.26 | 27.19 | 27.7 | 2 [symbol] |
| [symbol] ODAL | 29.31 | 25.46 | 25.13 | 25.68 | 25.2 | 2 [symbol] |

[symbol]

indicates data missing or illegible when filed

Table 8 shows the Cq values obtained with MS-qPCR reactions of D1b and D1b methylated with aHE methyltransferase. The MS-qPCR reactions yield a positive signal (i.e. a Cq value) when targets are methylated at the CpG dinucleotides present within the primers and probes. Some of the targets such as NODAL and HOXD3c could be detected from both unmethylated and aHE methylated D1b DNA while others such as AOX1, EPHX3, and NEUROG3 were only recovered from aHE methylated DNA under the bisulfite and amplification conditions used in this example. The amplification from the aHE methylated DNA was more reproducible as the difference in Cq values between replicas was smaller than that of the unmethylated DNA. The lower Cq values obtained with the methylated DNA also reflect a higher target copy number following bisulfite treatment (i.e. better analytical sensitivity). The in vitro methylation of the DNA enabled the analysis of multiple markers from limited amounts of DNA using a single bisulfite condition which couldn't be achieved with the unmodified DNA.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for analyzing the methylation status of at least one target sequence in a sample comprising:
providing a sample comprising DNA, wherein the DNA comprises at least one target sequence;
contacting the sample or purified DNA or tagged DNA with at least one methyltransferase that methylates non-cytosine nucleotides; and
assaying the sample or purified DNA or tagged DNA for the methylation status of one or more cytosine nucleotides in the at least one target sequence.

2. The method of claim 1, wherein the methylation status of at least two target sequences are analyzed.

3. The method of claim 1, wherein the sample is contacted with at least one methyltransferase that methylates non-cytosine nucleotides and at least one methyltransferase that methylates cytosine nucleotides.

4. The method of claim 1, wherein the at least one methyltransferase includes at least one methyltransferase that methylates adenine nucleotides present in the at least one target sequence.

5. The method of claim 1, wherein the at least one methyltransferase includes at least one methyltransferase that methylates adenine residues within a recognition sequence that comprises the sequence AG, RAR, GATC, GTAC, TCGA, CATG, AATT, GAWTC, SATC, AACCA, ACATC, GAATTC, GACGTC, CACAG, or any combination thereof, or
wherein the at least one methyltransferase is M.EcoKDam, M.CviQ1, M.CviQX1, M.CvQII, M.TagI, M.Tsp509I, M.AatII, M.BceJI, M.EcoR1, M.EcoGII or any combination thereof.

6. The method of claim 1, wherein the at least one methyltransferase includes at least one methyltransferase that methylates adenine nucleotides without a specific recognition sequence.

7. The method of claim 1, wherein the method further comprises purifying the DNA from the sample to thereby produce purified DNA.

8. The method of claim 1, wherein the at least one methyltransferase methylates at least 0.3% of nucleotides in the sample.

9. The method of claim 1, wherein the at least one methyltransferase methylates at least 0.8% of nucleotides in the sample.

10. (canceled)

11. The method of claim 1, wherein the at least one methyltransferase methylates at least 5% of nucleotides in the sample.

12. The method of claim 3, wherein the cytosine methyltransferase methylates cytosine residues within a recognition sequence that comprises the sequence AGCT, GGCC, GCGC, GTAC, GATC, TCGA, CCGG, GCNGC, CCWGG, RCATGY, GAGCTC, RGCB, CCD, CGR, GC, CC, or any combination thereof, or
wherein the at least one methyltransferase is M.AluI, M.HaeIII, M.HhaI, M.RsaI, M.Sau3AI, M.EsaLHCI, M.EsaBC2I, M.HpaII, M.MspI, M.EcoKDcm, M.BamHI, M.CviPI, M.CviPII, M.CviQIX, M.CviQVIII, M.CviQX or any combination thereof.

13. (canceled)

14. A method for quantitating the methylation status of at least one target sequence present in a sample to quantitate the percentage of the at least one target sequence that is methylated, comprising the method of claim 1, and further comprising:
comparing the amount of methylated and unmethylated cytosine nucleotides in the at least one target sequence to a corresponding amount in a standard.

15. The method of claim 14, wherein the standard is generated from known amounts of methylated and unmethylated DNA.

16. A method for analyzing the density of methylation of at least one target sequence present in a sample to improve the quantitation of the methylation of two or more cytosines within the at least one target sequence comprising the method of claim 14, wherein the methylation status of two or more cytosine nucleotides in the at least one target sequence is determined during the assay step.

17. The method of claim 1, wherein the assay step comprises digesting the sample or purified DNA or tagged DNA with a sequence specific restriction endonuclease.

18. The method of claim 1, wherein the assay step comprises targeted sequencing, next generation sequencing or direct sequencing.

19. The method of claim 1, wherein the assay step comprises using single nucleotide primer extension, fluorescent-based quantitative PCR, headloop suppression PCR, ligation-mediated amplification, microarray analysis, bead hybridization, flow cytometry, mass spectrometry, or any combination thereof.

20. (canceled)

21. The method of claim 1, wherein the sample is a tissue obtained during a biopsy or a surgical resection.

22. The method of claim 1, wherein the sample is a bodily fluid.

23. The method of claim 22, wherein the bodily fluid is blood, blood plasma, blood serum, urine, sputum, ejaculate, semen, prostatic fluid, tears, sweat, saliva, lymph fluid, bronchial lavage, pleural effusion, peritoneal fluid, meningeal fluid, amniotic fluid, glandular fluid, fine needle aspirates, nipple aspirate fluid, spinal fluid, conjunctival fluid, vaginal fluid, duodenal juice, pancreatic juice, pancreatic ductal epithelium, pancreatic tissue bile, cerebrospinal fluid, or any combination thereof.

24. The method of claim 1, wherein the sample comprises fragments of fewer than 150 contiguous nucleotides, wherein the fragments include the at least one target sequence.

25. The method of claim 1, wherein the sample includes less than 15 ng of DNA.

26. A method for analyzing the methylation status of at least one target sequence present in a sample comprising:
providing a sample comprising RNA, wherein the RNA comprises at least one target sequence;
contacting the sample or purified RNA or tagged RNA with at least one methyltransferase that methylates non-cytosine nucleotides; and
assaying the sample or purified RNA or tagged RNA for the methylation status of one or more cytosine nucleotides in the at least one target sequence.

27. The method of claim 1, wherein the method further comprises ligating a linker to the DNA to thereby produce tagged DNA.

28. The method of claim 1, wherein the method further comprises contacting the sample with at least one methyltransferase that methylates cytosine nucleotides.

29. The method of claim 1, wherein the assaying step comprises using a chemical and/or enzymes to convert cytosine nucleotides into uracil nucleotides.

30. The method of claim 29, wherein the chemical is a bisulfite salt.

31. The method of claim 1, wherein the assaying step comprises using a chemical and/or enzymes to convert unmethylated cytosine nucleotides into uracil nucleotides.

32. The method of claim 26, wherein the method further comprises purifying the RNA from the sample to thereby produce purified RNA.

33. The method of claim 26, wherein the method further comprises ligating a linker to the RNA to thereby produce tagged RNA.
